SEQUENCE LISTING (8 sequences)

SEQ ID NO: 1 - DNA, 1378 bp, Homo sapiens
  source 1..1378: mol_type = genomic DNA
agactggagagaagcaggcctgagcctccccaaaggcagctcctggggactcccaggaccacaggctgagacgagacgcagggtggctggaggaagtgagaggtgaactcagcctgggactggctgggcgagactctccacctgctccctgggaccatcgcccaccatggctgtggcccagcagctgcgggccgagagtgactttgaacagcttccggatgatgttgccatctcggccaacattgctgacatcgaggagaagagaggcttcaccagccactttgttttcgtcatcgaggtgaagacaaaaggaggatccaagtacctcatctaccgccgctaccgccagttccatgctttgcagagcaagctggaggagcgcttcgggccagacagcaagagcagtgccctggcctgtaccctgcccacactcccagccaaagtctacgtgggtgtgaaacaggagatcgccgagatgcggatacctgccctcaacgcctacatgaagagcctgctcagcctgccggtctgggtgctgatggatgaggacgtccggatcttcttttaccagtcgccctatgactcagagcaggtgccccaggcactccgccggctccgcccgcgcacccggaaagtcaagagcgtgtccccacagggcaacagcgttgaccgcatggcagctccgagagcagaggctctatttgacttcactggaaacagcaaactggagctgaatttcaaagctggagatgtgatcttcctcctcagtcggatcaacaaagactggctggagggcactgtccggggagccacgggcatcttccctctctccttcgtgaagatcctcaaagacttccctgaggaggacgaccccaccaactggctgcgttgctactactacgaagacaccatcagcaccatcaaggacatcgcggtggaggaagatctcagcagcactcccctattgaaagacctgctggagctcacaaggcgggagttccagagagaggacatagctctgaattaccgggacgctgagggggatctggttcggctgctgtcggatgaggacgtagcgctcatggtgcggcaggctcgtggcctcccctcccagaagcgcctcttcccctggaagctgcacatcacgcagaaggacaactacagggtctacaacacgatgccatgagctgacggtgtccctggagcagtgaggggacaccagcaaaaaccttcagctctcagaggagattgggaccaggaaaacctgggaggatgggcagacttcctgtctttgaggctaatggacccgtggggcttgtaatctgtctctttctactatttacatctgatttaaataaaccattccatctgaaagggg

SEQ ID NO: 2 - protein, 339 aa, Homo sapiens
MAVAQQLRAESDFEQLPDDVAISANIADIEEKRGFTSHFVFVIEVKTKGGSKYLIYRRYRQFHALQSKLEERFGPDSKSSALACTLPTLPAKVYVGVKQEIAEMRIPALNAYMKSLLSLPVWVLMDEDVRIFFYQSPYDSEQVPQALRRLRPRTRKVKSVSPQGNSVDRMAAPRAEALFDFTGNSKLELNFKAGDVIFLLSRINKDWLEGTVRGATGIFPLSFVKILKDFPEEDDPTNWLRCYYYEDTISTIKDIAVEEDLSSTPLLKDLLELTRREFQREDIALNYRDAEGDLVRLLSDEDVALMVRQARGLPSQKRLFPWKLHITQKDNYRVYNTMP

SEQ ID NO: 3 - DNA, 1463 bp, Mus musculus
  source 1..1463: mol_type = genomic DNA
tgctgtgcagctgggaagtggctgagaagacgtgtgagggccaggcagctcaaagcctgaaggaggtacttcaaggagggtccagagaagctgggcagggcgggacctcctcaaaggctgcccctggggacaccccaagcttcaggaagctctcagatgcacataggaggaagtgagaggtgaactcggcctggatctggttgggcaaggcccccatccatctgcttttctgactacccacagccatggccctggcccagcagctgcgatcagagagcgactttgagcagcttccagacgatgtggccgtctcagccaacatcgctgacatcgaggagaagaggggcttcaccagccactttgtttttgtcatcgaggtcaaaacaaaaggagggtccaagtatctcatctaccgccgctatcgccagttctacgccctgcagagcaagctggaggagcggtttgggccagagagcaagaacagccctttcacctgcagcctgcccacattgccagccaaagtctacatgggcgcaaaacaagagatcgctgagactcggatcccggccctcaatgcctacatgaagaacctcctgagcctgccggtctgcgtgctgatggaccccgacgtcaggatcttcttctatcagtctgcatacgatgctgagcaggtgccccaggcactccgcaggctccgaccgcgcacgcgcaaaatcaagggtgtgtctccacaaggggccatcatggatcgcatggaagcgccaagagcagaggccttgtttgacttcactgggaacagcaaattggagctaagtttcaaagctggagatgtgatcttccttctcagtaagatcaacaaagactggctggagggcacctcccagggagccacaggcatcttcccagggtccttcgtgaagatccttaaggactttcccgaggacgaggacaccaccaactggctacgatgctacttctatgaagacacaggcaaaaccatcaaggacattgcggtggaggaggacctcagcagcacgcccctgttcaaagacctgctagcgctcatgaggcgtgagttccagagagaagacattgcccttagctaccaggatgctgagggggacttggttaggctgctgtcagatgaggatgtgggactcatggtgaagcaagcccgaggcctcccttcccagaagcgcctcttcccctggaagctgcatgtcacacagaaggacaactacagtgtctacaacactgtcccctgagtcacagggaccctgtggcaaggacggggcgctcagcaaagggcctttaccttccaagaacacggagaccttcagggagactgatgtaagatgcaaacctcctggcccagagtgcctactggacaggattctattaagtttttgacactctaccttttggattcaataaaccattctgtctctaagggcaaaaaaaaa

SEQ ID NO: 4 - protein, 339 aa, Mus musculus
MALAQQLRSESDFEQLPDDVAVSANIADIEEKRGFTSHFVFVIEVKTKGGSKYLIYRRYRQFYALQSKLEERFGPESKNSPFTCSLPTLPAKVYMGAKQEIAETRIPALNAYMKNLLSLPVCVLMDPDVRIFFYQSAYDAEQVPQALRRLRPRTRKIKGVSPQGAIMDRMEAPRAEALFDFTGNSKLELSFKAGDVIFLLSKINKDWLEGTSQGATGIFPGSFVKILKDFPEDEDTTNWLRCYFYEDTGKTIKDIAVEEDLSSTPLFKDLLALMRREFQREDIALSYQDAEGDLVRLLSDEDVGLMVKQARGLPSQKRLFPWKLHVTQKDNYSVYNTVP

SEQ ID NO: 5 - DNA, 4853 bp, Homo sapiens
  source 1..4853: mol_type = genomic DNA
actctgcctagaggctccagaagaagactggtctctcccaccacacagaggcctggaggaggcagaggccaggagggagaggtcccaagagcctgtgaaatgggtctggcctggctcccagctgggcaggaacacaggacttcaggacactaaggaccctgtcatgcccatggccagcacccaccagtgctggtgcctgcctgtccagagctgaccagggagatggtgctggcccaggggctgctctccatggccctgctggccctgtgctgggagcgcagcctggcaggggcagaagaaaccatcccgctgcagaccctgcgctgctacaacgactacaccagccacatcacctgcaggtgggcagacacccaggatgcccagcggctcgtcaacgtgaccctcattcgccgggtgaatgaggacctcctggagccagtgtcctgtgacctcagtgatgacatgccctggtcagcctgcccccatccccgctgcgtgcccaggagatgtgtcattccctgccagagttttgtcgtcactgacgttgactacttctcattccaaccagacaggcctctgggcacccggctcaccgtcactctgacccagcatgtccagcctcctgagcccagggacctgcagatcagcaccgaccaggaccacttcctgctgacctggagtgtggcccttgggagtccccagagccactggttgtccccaggggatctggagtttgaggtggtctacaagcggcttcaggactcttgggaggacgcagccatcctcctctccaacacctcccaggccaccctggggccagagcacctcatgcccagcagcacctacgtggcccgagtacggacccgcctggccccaggttctcggctctcaggacgtcccagcaagtggagcccagaggtttgctgggactcccagccaggggatgaggcccagccccagaacctggagtgcttctttgacggggccgccgtgctcagctgctcctgggaggtgaggaaggaggtggccagctcggtctcctttggcctattctacaagcccagcccagatgcaggggaggaagagtgctccccagtgctgagggaggggctcggcagcctccacaccaggcaccactgccagattcccgtgcccgaccccgcgacccacggccaatacatcgtctctgttcagccaaggagggcagagaaacacataaagagctcagtgaacatccagatggcccctccatccctcaacgtgaccaaggatggagacagctacagcctgcgctgggaaacaatgaaaatgcgatacgaacacatagaccacacatttgagatccagtacaggaaagacacggccacgtggaaggacagcaagaccgagaccctccagaacgcccacagcatggccctgccagccctggagccctccaccaggtactgggccagggtgagggtcaggacctcccgcaccggctacaacgggatctggagcgagtggagtgaggcgcgctcctgggacaccgagtcggtgctgcctatgtgggtgctggccctcatcgtgatcttcctcaccatcgctgtgctcctggccctccgcttctgtggcatctacgggtacaggctgcgcagaaagtgggaggagaagatccccaaccccagcaagagccacctgttccagaacgggagcgcagagctttggcccccaggcagcatgtcggccttcactagcgggagtcccccacaccaggggccgtggggcagccgcttccctgagctggagggggtgttccctgtaggattcggggacagcgaggtgtcacctctcaccatagaggaccccaagcatgtctgtgatccaccatctgggcctgacacgactccagctgcctcagatctacccacagagcagccccccagcccccagccaggcccgcctgccgcctcccacacacctgagaaacaggcttccagctttgacttcaatgggccctacctggggccgccccacagccgctccctacctgacatcctgggccagccggagcccccacaggagggtgggagccagaagtccccacctccagggtccctggagtacctgtgtctgcctgctggggggcaggtgcaactggtccctctggcccaggcgatgggaccaggacaggccgtggaagtggagagaaggccgagccagggggctgcagggagtccctccctggagtccgggggaggccctgcccctcctgctcttgggccaagggtgggaggacaggaccaaaaggacagccctgtggctatacccatgagctctggggacactgaggaccctggagtggcctctggttatgtctcctctgcagacctggtattcaccccaaactcaggggcctcgtctgtctccctagttccctctctgggcctcccctcagaccagacccccagcttatgtcctgggctggccagtggaccccctggagccccaggccctgtgaagtcagggtttgagggctatgtggagctccctccaattgagggccggtcccccaggtcaccaaggaacaatcctgtcccccctgaggccaaaagccctgtcctgaacccaggggaacgcccggcagatgtgtccccaacatccccacagcccgagggcctccttgtcctgcagcaagtgggcgactattgcttcctccccggcctggggcccggccctctctcgctccggagtaaaccttcttccccgggacccggtcctgagatcaagaacctagaccaggcttttcaagtcaagaagcccccaggccaggctgtgccccaggtgcccgtcattcagctcttcaaagccctgaagcagcaggactacctgtctctgcccccttgggaggtcaacaagcctggggaggtgtgttgagacccccaggcctagacaggcaaggggatggagagggcttgccttccctcccgcctgaccttcctcagtcatttctgcaaagccaaggggcagcctcctgtcaaggtagctagaggcctgggaaaggagatagccttgctccggcccccttgaccttcagcaaatcacttctctccctgcgctcacacagacacacacacacacacgtacatgcacacatttttcctgtcaggttaacttatttgtaggttctgcattattagaactttctagatatactcattccatctccccctcatttttttaatcaggtttccttgcttttgccatttttcttccttcttttttcactgatttattatgagagtggggctgaggtctgagctgagccttatcagactgagatgcggctggttgtgttgaggacttgtgtgggctgcctgtccccggcagtcgctgatgcacatgacatgattctcatctgggtgcagaggtgggaggcaccaggtgggcacccgtgggggttagggcttggaagagtggcacaggactgggcacgctcagtgaggctcagggaattcagactagcctcgattgtcactccgagaaatgggcatggtattgggggtcgggggggcggtgcaagggacgcacatgagagactgtttgggagcttctggggagccctgctagttgtctcagtgatgtctgtgggacctccagtcccttgagaccccacgtcatgtagagaagttaacggcccaagtggtgggcaggctggcgggacctggggaacatcaggagaggagtccagagcccacgtctactgcggaaaagtcaggggaaactgccaaacaaaggaaaatgccccaaaggcatatatgctttagggcctttggtccaaatggcccgggtggccactcttccagatagaccaggcaactctccctcccaccggccacagatgaggggctgctgatctatgcctgggcctgcaccagggattatggttcttttaaatctttgcctttcagatacaggaaaaataatggcattaaattgctttaatttgcattattttagttatccagtttgcacatatttttataggtatcttaggcatcgattggtattttttaactgggccaagcccattaaggtctttcttctgttgggtgctatcattttctgattaagtctttttgactattgacatacagtctttcacagatggtggagtgtttttcccccaaatctgttgtttgtcttataatgttgtatatgaggttttatggtgtatgaatatgaatgcttctgtaatgtcaaacagatccctagtaaactccttcttcacttttactgtcagatttacaaaggtcctcccattgcaaagcagtgtttgtcctaatttatatattgtttttctagttcattttgtgtttccaacttttcatgtaaaattttaattatttttgaatgtgtggatgtgagactgaggtgccttttggtactgaaattctttttccatgtacctgaagtgttacttttgtgatataggaaatccttgtatatatactttattggtccctaggcttcctattttgttaccttgctttctctatggcatccaccattttgattgttctacttttatgatatgttttcataagtggttaagcaagtattctcgttacttttgctcttaaatccctattcattacagcaatgttggtggtcaaagaaaatgataaacaacttgaatgttcaatggtcctgaaatacataacaacattttagtacattgtaaagtagaatcctctgttcataatgaacaagatgaaccaatgtggattagaaagaagtccgagatattaattccaaaatatccagacattgttaaagggaaaaaattgcaataaaatatttgtaacataaaa

SEQ ID NO: 6 - protein, 897 aa, Homo sapiens
MVLAQGLLSMALLALCWERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESVLPMWVLALIVIFLTIAVLLALRFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVC

SEQ ID NO: 7 - DNA, 550 bp, Homo sapiens
  source 1..550: mol_type = genomic DNA
acttgctctgcccgctcaaacagttgcaggatgtcgatgacagacttgctgaacgctgaggacatcaagaaggcggtgggagcctttagcgctaccgactccttcgaccacaaaaagttcttccaaatggtcggcctgaagaaaaagagtgcggatgatgtgaagaaggtgtttcacatgctggacaaggacaaaagtggcttcatcgaggaggatgagctgggattcatcctaaaaggcttctccccagatgccagagacctgtctgctaaagaaaccaagatgctgatggctgctggagacaaagatggggacggcaaaattggggttgacgaattctccactctggtggctgaaagctaagaagcactgactgcccctggtcttccacctctctgccctgaacacccaatctcggcccctctcgccaccctcctgcatttctgttcagttcgtttatgttattttttactcccccatcccctgtggccctctaatgacaccattcttctggaaaatgctggagaagcaataaaggttgtaccagtca

SEQ ID NO: 8 - protein, 110 aa, Homo sapiens
MSMTDLLNAEDIKKAVGAFSATDSFDHKKFFQMVGLKKKSADDVKKVFHMLDKDKSGFIEEDELGFILKGFSPDARDLSAKETKMLMAAGDKDGDGKIGVDEFSTLVAES

(19) United States
(12) Patent Application Publication
(10) Pub. No.: US 2025/0213683 A1
(43) Pub. Date: Jul. 3, 2025

(54) COMBINATION IMMUNE CHECKPOINT INHIBITOR THERAPIES

(71) Applicant: The Jackson Laboratory, Bar Harbor, ME (US)

(72) Inventor: Edison T. Liu, Bar Harbor, ME (US)

(73) Assignee: The Jackson Laboratory, Bar Harbor, ME (US)

(21) Appl. No.: 18/852,755

(22) PCT Filed: Mar. 30, 2023
(86) PCT No.: PCT/US2023/065153

§ 371 Date: Sep. 30, 2024

Related U.S. Application Data
(60) Provisional application No. 63/326,317, filed on Apr. 1, 2022.

(51) Int. Cl. A61K 39/395; A61P 35/00; C12Q 1/6886

(52) U.S. Cl. CPC A61K 39/39558; A61P 35/00; C12Q 1/6886; C12Q 2600/106; C12Q 2600/156; C12Q 2600/158

(57) ABSTRACT

Provided herein are methods comprising administering to a cancer patient an agent that modifies expression of a gene or function of a product encoded by the gene, wherein the gene is a human ortholog of a gene that maps to a mouse Chromosome 15 quantitative trait locus (QTL), for example, the human NCF4 gene.

### GOVERNMENT LICENSE RIGHTS

This invention was made with government support under CA034196 awarded by the National Cancer Institute of the National Institutes of Health. The government has certain rights in the invention.

### RELATED APPLICATIONS

This application claims the benefit under 35 U.S.C. § 119 (e) of U.S. provisional application number 63,326,317, filed Apr. 1, 2022, which is incorporated by reference herein in its entirety.

### REFERENCE TO AN ELECTRONIC SEQUENCE LISTING

The contents of the electronic sequence listing (J022770113WO00-SEQ-EAS; Size: 17,821 bytes; and Date of Creation: Mar. 30, 2023) is herein incorporated by reference in its entirety.

### BACKGROUND

One approach to cancer immunotherapy is checkpoint inhibition, which is showing progress in both clinical and preclinical studies as an adjuvant and alternative to traditional cancer therapies. The presence of T cells and other lymphocytes in the tumor microenvironment is highly correlated with an improved outcome. T cells, however, have a set of cell surface receptors termed immune checkpoints that when activated suppress T cell function. Upregulation of these immune checkpoint receptors, such as programmed cell death 1 (PD-1) and cytotoxic T lymphocyte associated protein 4 (CTLA-4), occurs during T cell activation in an effort to prevent damage from an excessive immune response. The efficacy of checkpoint inhibition results from releasing T cells from the inhibitory effects of checkpoint molecules. Thus, immune checkpoint inhibitors allow the adaptive immune system to respond to tumors more effectively. While there has been clinical success in treating different types of cancer by blocking immune checkpoint receptors, resistance to immunotherapy and relapse remain a concern.

### SUMMARY

The present disclosure provides, in some aspects, promising therapeutic gene targets, the modulation of which may overcome resistance to cancer immunotherapy in certain patient populations. The underlying studies described herein were designed in part to dissect the genetic control of a cancer patient's response to immune checkpoint inhibitors using an established panel of Collaborative Cross (CC) mouse strains for tumor engraftment. By establishing a quantitative means for assessing immune checkpoint inhibitor (ICI) response across all models tested, and establishing heritability of the ICI (e.g., anti-PD1) response, the studies described herein demonstrate, for example, that genetic background affects ICI efficacy (e.g., anti-PD1 ICI efficacy) across multiple tumor types. The studies provided herein also support an association between immunophenotypic variability within genetic background and ICI (e.g., anti-PD1) response.

Some aspects of the present disclosure provide a method, comprising administering to a cancer patient an agent that modifies expression of a gene or function of a product of the gene, wherein the gene is a homolog (e.g., human ortholog) of a gene that maps to a mouse Chromosome 15 quantitative trait locus (QTL).

Other aspects of the present disclosure provide a method, comprising: (a) assaying a biological sample from a cancer patient for presence of a gene or a product of the gene, or a variant of the gene or product of the gene, wherein the gene maps to a Chromosome 15 quantitative trait locus (QTL); and (b) administering to the cancer patient an agent that modifies expression of the gene or function of the product of the gene, or modifies expression of the variant of the gene or function of the variant of the product of the gene.

In some embodiments, the method further comprises administering to the cancer patient an immune checkpoint inhibitor.

Yet aspects of the present disclosure provide a method, comprising: (a) administering to a cancer patient an agent that modifies expression of a gene or function of the gene product, wherein the gene maps to a Chromosome 15 quantitative trait locus (QTL); and (b) administering to the cancer patient an immune checkpoint inhibitor (ICI).

In some embodiments, the biological sample is a blood sample. In some embodiments, the biological sample comprises monocytes and macrophages.

In some embodiments, the agent is administered only if expression of the gene or function of the gene is higher. In some embodiments, the agent is administered only if expression of the gene or function of the gene is higher in monocytes and macrophages, relative to a control.

In some embodiments, the agent inhibits expression of the gene or function of the product of the gene, or inhibits expression of the variant of the gene or function of the variant of the product of the gene. In some embodiments, the agent targets monocytes and macrophages. As described herein, “responders” to ICI therapy were shown to have lower levels of gene expression at the Chromosome 15 QTL locus (e.g., NCF4 gene expression) than non-responders.

In some embodiments, the Chromosome 15 QTL comprises an NCF4 gene.

In some embodiments, the Chromosome 15 QTL spans a chromatin region within 2 megabases (Mb) upstream from and 2 Mb downstream from the NCF4 gene.

In some embodiments, the gene is an NCF4 gene or NCF4 gene variant.

In some embodiments, the NCF4 gene variant encodes an NCF4 protein variant that comprises a mutation corresponding to an S85N mutation in a wild-type mouse NCF4 protein.

In some embodiments, the gene is a CSF2RB gene or a CSF2RB gene variant.

In some embodiments, the gene is a PVALB gene or a PVALB gene variant.

In some embodiments, the agent is an inhibitor of NCF4, CSF2RB, or PVALB gene expression and/or NCF4, CSF2RB, or PVALB protein function. In some embodiments, the agent is an inhibitor of NCF4 gene expression and/or NCF4 protein function.

In some embodiments, the agent is a direct inhibitor of NCF4, CSF2RB, or PVALB gene expression and/or NCF4, CSF2RB, or PVALB protein function. In some embodiments, the agent is a direct inhibitor of NCF4 gene expression and/or NCF4 protein function.

In some embodiments, the agent is an anti-p40-phox antibody.

In some embodiments, the agent is selected from antibodies, small molecule drug, and gene editing molecules.

In some embodiments, the immune checkpoint inhibitor is selected from programmed cell death protein 1/programmed cell death ligand 1 (PD-1/PD-L1) inhibitors, cytotoxic T lymphocyte-associated antigen-4 (CTLA-4) inhibitors, lymphocyte activation gene-3 (LAG-3) inhibitors, T cell immunoglobulin and mucin-domain containing-3 (TIM-3) inhibitors, T cell immunoglobulin and ITIM domain (TIGIT) inhibitors, and V-domain Ig suppressor of T cell activation (VISTA) inhibitors.

In some embodiments, the immune checkpoint inhibitor is a PD-1/PD-L1 inhibitor.

In some embodiments, the PD-1/PD-L1 inhibitor is an anti-PD-1 antibody or an anti-PD-L1 antibody.

In some embodiments, the cancer patient has a basal cell carcinoma, bladder cancer, breast cancer, cervical cancer, colorectal cancer, endometrial cancer, esophageal carcinoma, gastric cancer, head and neck cancer, hepatocellular carcinoma, Hodgkin's lymphoma, malignant pleural mesothelioma, melanoma, melanoma, metastatic, merkel cell carcinoma, non-small cell lung cancer, primary mediastinal large b-cell lymphoma, renal cell carcinoma, small cell lung cancer, solid tumors, squamous cell carcinoma, stomach cancer, or a urothelial carcinoma.

Some aspects of the present disclosure provide a method, comprising: selecting a subject who is a candidate for immune checkpoint inhibitory (ICI) therapy; obtaining a biological sample from the subject; assaying the biological sample for a gene or protein level one or more biomarkers selected from RGS1, NKG7 and CCL5; and optionally comparing the gene or protein level to a control level, wherein the control level is based on a biological sample that is non-responsive to ICI therapy.

In some embodiments, the subject is a cancer patient.

In some embodiments, the ICI therapy is anti-PD-L1 or anti-PD-1 antibody therapy.

In some embodiments, the biological sample is a blood sample.

In some embodiments, the method further comprises diagnosing the subject as a responder to ICI therapy if the gene or protein level of one or more of RGS1, NKG7 and CCL5 is at least 1-fold, 1.1-fold, 1.2-fold, 1.3-fold, 1.4-fold, or 1.5-fold higher than the control level.

In some embodiments, the method further comprises administering an ICI therapy to the subject.

### BRIEF DESCRIPTION OF THE DRAWINGS

FIG. 1 shows a sampling of diversity of tumor growth and anti-PD1 responses observed across CCF1 lines in the MC38 tumor model.

FIG. 2 shows quantifiable modulation of anti-PD-1 response by using a novel rate-based treatment/control metric in the MC38 tumor model system.

FIG. 3 shows significant heritability of tumor growth rate in CT26, EMT6, and MC38 tumor models grown in diverse CCF1 host lines.

FIG. 4 shows a significant Quantitative Trait Loci (QTL) on Chromosome 15 (Chr15) by mapping of anti-PD-1 treated tumor growth rate in CCF1 mice.

FIG. 5 shows that Ncf4 exhibits both high expression and large difference between responder and non-responder strain tumors.

FIG. 6 shows Ncf4 difference is driven mostly by gene expression and not by difference in cell numbers.

FIG. 7 shows candidate causal variants near Ncf4 with potential for effects on gene regulation or protein function.

FIG. 8 shows a survival curve comparing C57BL/6J and BALB/cJ F1 strains treated with anti-PD-1.

FIG. 9 shows signatures consistent with putative Cxc19⁺ (PD-L1^{high}MHCII^{high}) macrophages and CTLs enriched in responder tumors.

FIG. 10 shows enrichment of intratumor CTLs and putative Cxc19⁺ (PD-L1^{high}MHCII^{high}) macrophages in responders.

FIG. 11 shows that Cxcl9 and Ifng are prominent biomarkers of immune checkpoint response.

FIG. 12 shows that Cxcl9 and Ifng expression is significantly higher in responder strain MC38 tumors.

FIG. 13 shows that macrophage clusters 4 and 7, which express Cxc19 and are enriched in responder tumors.

FIG. 14 shows that response to IFNγ GO gene set expression is most expressed by cluster 7.

FIG. 15 shows a working model, based on the evidence provided herein, that genetics may control ICI efficacy through intratumor macrophage differentiation.

FIG. 16 shows a model of response/non-response in the MC38 IO model. In responder tumors (left), more Cxc19^{high}PD-L1^{high} monocyte/macrophages and higher CXCL9 expression recruits more tumor killing CTL. These CTL ultimately begin to display an “exhausted” phenotype and are suppressed by Cxc19^{high}PD-L1^{high} monocyte/macrophages through CTL PD-1 expression. Upon administration of anti-PD1 ICI capable of disrupting PD-L1-PD1 interactions, an anti-tumor immune response is unleashed, and tumor regression occurs.

FIG. 17 shows Visium transcriptomics data from the MC38 IO model. Despite similar pan-immune cell infiltration (CD45), responder tumors (CC75 F1) have more Cxcl9⁺ monocyte/macrophages and CTL. These CTL co-localize with macrophages and dendritic cells to a higher degree in responder (CC75 F1) versus non-responder (CC80 F1) tumors, with Cxcl9⁺ macrophage-CTL co-localization predominating.

### DETAILED DESCRIPTION

The present disclosure is based at least in part on data resulting from studies profiling CCF1 mouse lines for a response to anti-PD-1 treatment and determining the effect of host genetics on ICI response. The quantitative and reproducible variation in anti-PD1 response across multiple CCF1 lines in various syngeneic mouse tumor models support the central hypothesis that genetic background of the host significantly affects the anti-PD-1 response.

Genetic mapping of anti-PD-1 response in CCF1 lines was also conducted in order to identify genomic loci (quantitative trait loci; QTL) harboring variation that influences this phenotype. A QTL was mapped for responses in syngeneic tumor lines, and a significant QTL (permutation P<0.05) was found for a response in MC38 on Chromosome 15 (Chr15). Extensive investigation of the Chr15 QTL has been performed in the MC38 model. The QTL interval implicated by genetic mapping spans 5.2 megabases (Mb) and contains approximately 200 genes. Genetic results and functional genomics data have been integrated in order to identify the causal gene or genes, as well as individual candidate genetic variants, likely responsible for driving this QTL.

Single cell transcriptomics data was used to probe the expression of genes located within this Chr15 QTL interval. Only a subset of genes was expressed in the immunocytes, and of these genes, Ncf4 was identified as a gene that was highly expressed and had a large difference in expression between non-responder and responder strain tumors. This difference in Ncf4 expression appears to be largely driven by a difference in expression within monocytes and macrophages and not due to a greater number of these cells. Three Ncf4 coding variants were also identified along with several additional variants upstream of Ncf4 that lie within open chromatin regions containing computationally predicted motif binding sites. These variants lie within a cluster in an ˜50 kb region near the middle of the Chr15 QTL. These variants constitute candidates that may affect gene regulation or protein function of Ncf4 and could thereby modulate anti-PD-1 response.

Ncf4 is a member of the NADPH oxidase complex, which is a major source of cellular reactive oxygen species. Further, NADPH oxidase impacts antigen presentation, the Ncf4 has a role in innate immunity and human disease as well as associations with kidney and colorectal cancer. Thus, the functional analysis of the host immune cells in model tumors and the genetic mapping described herein show that Ncf4 is highly plausible candidate gene for modulating the anti-PD-1 response.

Other genes of interest in this Chr15 QTL include Csf2rb (GM-CSF receptors) and Pvalb.

### Quantitative Trait Loci

Quantitative trait locus (QTL) analysis is a statistical method that links two types of information—phenotypic data (trait measurements) and genotypic data (usually molecular markers)—in an attempt to explain the genetic basis of variation in complex traits (Falconer & Mackay, 1996; Kearsey, 1998; Lynch & Walsh, 1998). QTL analysis enables researchers to link certain complex phenotypes to specific regions of chromosomes. The goal of this process is to identify the action, interaction, number, and precise location of these regions (Miles & Wayne, Nature Education 2008; 1 (1): 208).

In general, a QTL analysis in mice, requires (1) two or more parental strains of mice that differ genetically with regard to the trait of interest, and (2) genetic markers that distinguish between these parental strains. Molecular markers are preferred for genotyping, because these markers are unlikely to affect the trait of interest. Several types of markers are used, including single nucleotide polymorphisms (SNPs), simple sequence repeats (SSRs, or microsatellites), restriction fragment length polymorphisms (RFLPs), and transposable element positions (Casa et al., 2000; Vignal et al., 2002; Gupta & Rustgi, 2004; Henry, 2006). To perform the QTL analysis, phenotypes and genotypes of a genetically heterogeneous population are scored. Markers that are genetically linked to a QTL influencing the trait of interest will segregate more frequently with trait values (large or small egg size in our example), whereas unlinked markers will not show significant association with phenotype (Miles & Wayne, Nature Education 2008; 1 (1): 208). Although QTL mapping approaches commonly employ schemes such as the F2 intercross or backcross, the present work utilized the Collaborative Cross (CC), a recombinant inbred line genetic resource population. In this work individual CC lines mated to a common strain served as the genetically and phenotypically variable mapping population.

In some embodiments, the methods provided herein comprise administering to a subject (e.g., a cancer patient) an agent that modifies expression of a gene or function of a product of the gene, wherein the gene is a human homolog of a gene that maps to a mouse Chr15 QTL. A homolog is a gene related to a second gene by descent from a common ancestral DNA sequence. The term, homolog, applies to the relationship between genes separated by the event of speciation (ortholog) or to the relationship between genes separated by the event of genetic duplication (paralog). Thus, a human ortholog of a gene that maps to a mouse chromosome 15 QTL is a human gene that maps to a syntenic region on human chromosome 8 or human chromosome 22. See, e.g., Nature 2002; 420:520-562, and website: informatics.jax.org/homology.shtml for discussion of human orthology.

In some embodiments, the mouse Chr15 QTL described herein spans a ˜5 Mb region mapping to the mouse genome, for example, GRCm38/mm10 Chr15: 75.79-81.31 Mb (see, e.g., PMID: 19468303 and website: ncbi.nlm.nih.gov/assembly/GCF_000001635.20). Thus, in some embodiments, a gene to be modified in accordance with the present disclosure is a human homolog (e.g., human ortholog) of a gene that maps to GRCm38/mm10 Chr15: 75.79-81.31 Mb. In some embodiments, the gene is a human homolog (e.g., human ortholog) of a gene that maps to a subregion within GRCm38/mm10 Chr15: 75.79-81.31 Mb. In some embodiments, the gene is a human homolog (e.g., human ortholog) of a gene that maps to GRCm38/mm10 Chr15: 78,175,584-78,274,852 Mb. In other embodiments, the gene is a human homolog (e.g., human ortholog) of a gene that maps to GRCm38/mm10 Chr15: 79,998,008-80,088,247 Mb.

### NCF4

In some embodiments, the Chromosome 15 QTL comprises an NCF4 gene. Thus, some aspects of the present disclosure provide methods that comprise administering to a subject (e.g., a cancer patient) an agent that modifies expression of the human neutrophil cytosolic factor 4 (NCF4) gene or modifies the function of the gene product. The NCF4 gene is located on human Chromosome 22 (22q12.3; Chr22: 36861006-36878015). Below is a non-limiting example of a human NCF4 gene and a human NCF4 protein sequence:

(SEQ ID NO: 1)

agactggaga gaagcaggcc tgagcctccc caaaggcagc
tcctggggac tcccaggacc acaggctgag acgagacgca
gggtggctgg aggaagtgag aggtgaactc agcctgggac
tggctgggcg agactctcca cctgctccct gggaccatcg
cccaccatgg ctgtggccca gcagctgcgg gccgagagtg
actttgaaca gcttccggat gatgttgcca tctcggccaa
cattgctgac atcgaggaga agagaggctt caccagccac
tttgttttcg tcatcgaggt gaagacaaaa ggaggatcca
agtacctcat ctaccgccgc taccgccagt tccatgcttt
gcagagcaag ctggaggagc gcttcgggcc agacagcaag
agcagtgccc tggcctgtac cctgcccaca ctcccagcca
aagtctacgt gggtgtgaaa caggagatcg ccgagatgcg
gatacctgcc ctcaacgcct acatgaagag cctgctcagc
ctgccggtct gggtgctgat ggatgaggac gtccggatct
tcttttacca gtcgccctat gactcagagc aggtgcccca
ggcactccgc cggctccgcc cgcgcacccg gaaagtcaag
agcgtgtccc cacagggcaa cagcgttgac cgcatggcag
ctccgagagc agaggctcta tttgacttca ctggaaacag
caaactggag ctgaatttca aagctggaga tgtgatcttc
ctcctcagtc ggatcaacaa agactggctg gagggcactg
tccggggagc cacgggcatc ttccctctct ccttcgtgaa
gatcctcaaa gacttccctg aggaggacga ccccaccaac
tggctgcgtt gctactacta cgaagacacc atcagcacca
tcaaggacat cgcggtggag gaagatctca gcagcactcc
cctattgaaa gacctgctgg agctcacaag gcgggagttc
cagagagagg acatagctct gaattaccgg gacgctgagg
gggatctggt tcggctgctg tcggatgagg acgtagcgct
catggtgcgg caggctcgtg gcctcccctc ccagaagcgc
ctcttcccct ggaagctgca catcacgcag aaggacaact
acagggtcta caacacgatg ccatgagctg acggtgtccc
tggagcagtg aggggacacc agcaaaaacc ttcagctctc
agaggagatt gggaccagga aaacctggga ggatgggcag
acttcctgtc tttgaggcta atggacccgt ggggcttgta
atctgtctct ttctactatt tacatctgat ttaaataaac
cattccatct gaaagggg

(SEQ ID NO: 2)

MAVAQQLRAE SDFEQLPDDV AISANIADIE EKRGFTSHFV
FVIEVKTKGG SKYLIYRRYR QFHALQSKLE ERFGPDSKSS
ALACTLPTLP AKVYVGVKQE IAEMRIPALN AYMKSLLSLP
VWVLMDEDVR IFFYQSPYDS EQVPQALRRL RPRTRKVKSV
SPQGNSVDRM AAPRAEALED FTGNSKLELN FKAGDVIFLL
SRINKDWLEG TVRGATGIFP LSFVKILKDF PEEDDPTNWL
RCYYYEDTIS TIKDIAVEED LSSTPLLKDL LELTRREFQR
EDIALNYRDA EGDLVRLLSD EDVALMVRQA RGLPSQKRLF
PWKLHITQKD NYRVYNTMP

Below is a non-limiting example of a mouse Ncf4 gene and a mouse NCF4 protein sequence:

(SEQ ID NO: 3)

tgctgtgcag ctgggaagtg gctgagaaga cgtgtgaggg
ccaggcagct caaagcctga aggaggtact tcaaggaggg
tccagagaag ctgggcaggg cgggacctcc tcaaaggctg
cccctgggga caccccaagc ttcaggaagc tctcagatgc
acataggagg aagtgagagg tgaactcggc ctggatctgg
ttgggcaagg cccccatcca tctgcttttc tgactaccca
cagccatggc cctggcccag cagctgcgat cagagagcga
ctttgagcag cttccagacg atgtggccgt ctcagccaac
atcgctgaca tcgaggagaa gaggggcttc accagccact
ttgtttttgt catcgaggtc aaaacaaaag gagggtccaa
gtatctcatc taccgccgct atcgccagtt ctacgccctg
cagagcaagc tggaggagcg gtttgggcca gagagcaaga
acagcccttt cacctgcagc ctgcccacat tgccagccaa
agtctacatg ggcgcaaaac aagagatcgc tgagactcgg
atcccggccc tcaatgccta catgaagaac ctcctgagcc
tgccggtctg cgtgctgatg gaccccgacg tcaggatctt
cttctatcag tctgcatacg atgctgagca ggtgccccag
gcactccgca ggctccgacc gcgcacgcgc aaaatcaagg
gtgtgtctcc acaaggggcc atcatggatc gcatggaagc
gccaagagca gaggccttgt ttgacttcac tgggaacagc
aaattggagc taagtttcaa agctggagat gtgatcttcc
ttctcagtaa gatcaacaaa gactggctgg agggcacctc
ccagggagcc acaggcatct tcccagggtc cttcgtgaag
atccttaagg actttcccga ggacgaggac accaccaact
ggctacgatg ctacttctat gaagacacag gcaaaaccat
caaggacatt gcggtggagg aggacctcag cagcacgccc
ctgttcaaag acctgctagc gctcatgagg cgtgagttcc
agagagaaga cattgccctt agctaccagg atgctgaggg
ggacttggtt aggctgctgt cagatgagga tgtgggactc
atggtgaagc aagcccgagg cctcccttcc cagaagcgcc
tcttcccctg gaagctgcat gtcacacaga aggacaacta
cagtgtctac aacactgtcc cctgagtcac agggaccctg
tggcaaggac ggggcgctca gcaaagggcc tttaccttcc
aagaacacgg agaccttcag ggagactgat gtaagatgca
aacctcctgg cccagagtgc ctactggaca ggattctatt
aagtttttga cactctacct tttggattca ataaaccatt
ctgtctctaa gggcaaaaaa aaa

(SEQ ID NO: 4)

MALAQQLRSE SDFEQLPDDV AVSANIADIE EKRGFTSHFV
FVIEVKIKGG SKYLIYRRYR QFYALQSKLE ERFGPESKNS
PFTCSLPTLP AKVYMGAKQE IAETRIPALN AYMKNLLSLP
VCVLMDPDVR IFFYQSAYDA EQVPQALRRL RPRTRKIKGV
SPQGAIMDRM EAPRAEALFD FTGNSKLELS FKAGDVIFLL
SKINKDWLEG TSQGATGIFP GSFVKILKDF PEDEDTTNWL
RCYFYEDTGK TIKDIAVEED LSSTPLFKDL LALMRREFQR
EDIALSYQDA EGDLVRLLSD EDVGLMVKQA RGLPSQKRLF
PWKLHVTQKD NYSVYNTVP

The protein encoded by this gene is a cytosolic regulatory component of the superoxide-producing phagocyte NADPH-oxidase, a multicomponent enzyme system important for host defense. This protein is preferentially expressed in cells of myeloid lineage. It interacts primarily with neutrophil cytosolic factor 2 (NCF2/p67-phox) to form a complex with neutrophil cytosolic factor 1 (NCF1/p47-phox), which further interacts with the small G protein RAC1 and translocates to the membrane upon cell stimulation. This complex then activates flavocytochrome b, the membrane-integrated catalytic core of the enzyme system. The PX domain of this protein can bind phospholipid products of the PI(3) kinase, which suggests its role in PI(3) kinase-mediated signaling events. The phosphorylation of this protein was found to negatively regulate the enzyme activity. Alternatively spliced transcript variants encoding distinct isoforms have been observed.

Described herein is compelling indirect evidence from the analysis of the Chr15 QTL that variation in and/or around Ncf4 is acting through this gene to modulate an anti-PD-1 response. Specifically, the responder genotype is associated with the less active Ncf4 allele (see Example 4 below).

In some embodiments, the Chr15 QTL spans a chromatin region within 2 Mb upstream from (5′ to) and/or 2 Mb downstream from (3′ to) the NCF4 gene. For example, the Chr15 QTL may span a chromatin region within 1 Mb upstream from and/or 1 Mb downstream from the NCF4 gene. This region includes, for example, Pvalb and Csf2rb2.

In some embodiments, the NCF4 gene is an NCF4 gene variant. In some embodiments, the NCF4 gene encodes an NCF4 protein variant. Variants are genes or proteins (including full length proteins and peptides) that differ in their respective nucleic acid or amino acid sequence relative to a wild-type, native, or reference sequence. A variant may possess one or more substitutions, deletions, and/or insertions at certain positions within its nucleic acid or amino acid sequence, as compared to a wild-type, native, or reference sequence. Ordinarily, variants have at least 75% identity to a wild-type, native or reference nucleic acid or amino acid sequence. In some embodiments, a variant has at least 80%, at least 85%, at least 90%, or at least 95% identity to a wild-type, native, or reference nucleic acid or amino acid sequence. In some embodiments, a variant differs from a wild-type, native or reference sequence by only one, two or three nucleotides or amino acids.

“Identity” refers to a relationship between two or among three or more sequences (e.g., amino acid sequences or nucleotide sequences) as determined by comparing the sequences to each other. Identity also refers to the degree of sequence relatedness between or among sequences as determined by the number of matches between or among strings of amino acids (polypeptides) or strings of nucleotides (polynucleotides). Identity is a measure of the percent of identical matches between the smaller of two or more sequences with gap alignments (if any) addressed by a particular mathematical model or computer program (e.g., “algorithms”). Identity of related polypeptides and polynucleotides can be readily calculated by known methods. “Percent (%) identity” as it applies to polypeptide or polynucleotide sequences is defined as the percentage of residues (amino acid or nucleic acid residues) in the candidate (first) polypeptide or polynucleotide sequence that are identical with the residues in a second polypeptide or polynucleotide sequence after aligning the sequences and introducing gaps, if necessary, to achieve the maximum percent identity.

Methods and computer programs for the alignment are well known in the art. It is understood that identity depends on a calculation of percent identity but may differ in value due to gaps and penalties introduced in the calculation. Generally, variants of a particular polynucleotide or polypeptide have at least 90%, 91%, 92%, 93%, 94%, 95%, 96%, 97%, 98%, 99% but less than 100% sequence identity to that particular wild-type, native, or reference sequence as determined by sequence alignment programs and parameters described herein and known to those skilled in the art. Such tools for alignment include but are not limited to those of the BLAST suite (Altschul, S. F., et al. Nucleic Acids Res. 1997; 25:3389-3402); and those based on the Smith-Waterman algorithm (Smith, T. F. & Waterman, M. S. J. Mol. Biol. 1981; 147:195-197). A general global alignment technique based on dynamic programming is the Needleman-Wunsch algorithm (Needleman, S. B. & Wunsch, C. D. J. Mol. Biol. 1920; 48:443-453). A Fast Optimal Global Sequence Alignment Algorithm (FOGSAA) also has been developed that purportedly produces global alignment of nucleotide and protein sequences faster than other optimal global alignment methods, including the Needleman-Wunsch algorithm.

In some embodiments, an NCF4 gene variant comprises a single nucleotide polymorphism (SNP). For example, a human NCF4 gene variant may encode a human NCF4 protein variant that comprises a mutation corresponding to an S85N mutation in a wild-type mouse NCF4 protein. “Corresponding mutations” can be determined, for example, by aligning a human NCF4 protein and a mouse NCF4 protein. For example, alignment of SEQ ID NO: 2 and SEQ ID NO: 4 shows that the S85N mutation in mouse NCF4 corresponds to a T85N mutation in human NCF4:

| human | MAVAQQLRAESDFEQLPDDVAISANIADIEEKRGFTSHFVFVIEVKTKGGSKYLIYRRYR | 60 |
|---|---|---|
| mouse | MALAQQLRSESDFEQLPDDVAVSANIADIEEKRGETSHFVFVIEVKTKGGSKYLIYRRYR | 60 |
| human | QFHALQSKLEERFGPDSKSSALACTLPTLPAKVYVGVKQEIAEMRIPALNAYMKSLLSLP | 120 |
| mouse | QFYALQSKLEERFGPESKNSPFTCSLPTLPAKVYMGAKQEIAETRIPALNAYMKNLLSLP | 120 |
| human | VWVLMDEDVRIFFYQSPYDSEQVPQALRRLRPRTRKVKSVSPQGNSVDRMAAPRAEALFD | 180 |
| mouse | VCVLMDPDVRIFFYQSAYDAEQVPQALRRLRPRTRKIKGVSPQGAIMDRMEAPRAEALFD | 180 |
| human | FTGNSKLELNFKAGDVIFLLSRINKDWLEGTVRGATGIFPLSFVKILKDFPEEDDPINWL | 240 |
| mouse | FTGNSKLELSFKAGDVIFLLSKINKDWLEGTSQGATGIFPGSFVKILKDFPEDEDTINWL | 240 |
| human | RCYYYEDTISTIKDIAVEEDLSSTPLLKDLLELTRREFQREDIALNYRDAEGDLVRLLSD | 300 |
| mouse | RCYFYEDTGKTIKDIAVEEDLSSTPLFKDLLALMRREFQREDIALSYQDAEGDLVRLLSD | 300 |
| (SEQ ID NO: 2) | | |
| human | EDVALMVRQARGLPSQKRLFPWKLHITQKDNYRVYNTMP | 339 |
| (SEQ ID NO: 4) | | |
| mouse | EDVGLMVKQARGLPSQKRLFPWKLHVTQKDNYSVYNTVP | 339 |

### CSF2RB

In some embodiments, the Chromosome 15 QTL comprises an CSF2RB gene. Thus, some aspects of the present disclosure provide methods that comprise administering to a subject (e.g., a cancer patient) an agent that modifies expression of the human Colony Stimulating Factor 2 Receptor Subunit Beta (CSF2RB) gene or modifies the function of the gene product. The CSF2RB gene is located on human Chromosome 22 (36,913,628-36,940,439; GRCh38: CM000684.2). Below is a non-limiting example of a human CSF2RB gene and a human CSF2RB protein sequence:

(SEQ ID NO: 5)

actctgccta gaggctccag aagaagactg gtctctccca
ccacacagag gcctggagga ggcagaggcc aggagggaga
ggtcccaaga gcctgtgaaa tgggtctggc ctggctccca
gctgggcagg aacacaggac ttcaggacac taaggaccct
gtcatgccca tggccagcac ccaccagtgc tggtgcctgc
ctgtccagag ctgaccaggg agatggtgct ggcccagggg
ctgctctcca tggccctgct ggccctgtgc tgggagcgca
gcctggcagg ggcagaagaa accatcccgc tgcagaccct
gcgctgctac aacgactaca ccagccacat cacctgcagg
tgggcagaca cccaggatgc ccagcggctc gtcaacgtga
ccctcattcg ccgggtgaat gaggacctcc tggagccagt
gtcctgtgac ctcagtgatg acatgccctg gtcagcctgc
ccccatcccc gctgcgtgcc caggagatgt gtcattccct
gccagagttt tgtcgtcact gacgttgact acttctcatt
ccaaccagac aggcctctgg gcacccggct caccgtcact
ctgacccagc atgtccagcc tcctgagccc agggacctgc
agatcagcac cgaccaggac cacttcctgc tgacctggag
tgtggccctt gggagtcccc agagccactg gttgtcccca
ggggatctgg agtttgaggt ggtctacaag cggcttcagg
actcttggga ggacgcagcc atcctcctct ccaacacctc
ccaggccacc ctggggccag agcacctcat gcccagcagc
acctacgtgg cccgagtacg gacccgcctg gccccaggtt
ctcggctctc aggacgtccc agcaagtgga gcccagaggt
ttgctgggac tcccagccag gggatgaggc ccagccccag
aacctggagt gcttctttga cggggccgcc gtgctcagct
gctcctggga ggtgaggaag gaggtggcca gctcggtctc
ctttggccta ttctacaagc ccagcccaga tgcaggggag
gaagagtgct ccccagtgct gagggggggg ctcggcagcc
tccacaccag gcaccactgc cagattcccg tgcccgaccc
cgcgacccac ggccaataca tcgtctctgt tcagccaagg
agggcagaga aacacataaa gagctcagtg aacatccaga
tggcccctcc atccctcaac gtgaccaagg atggagacag
ctacagcctg cgctgggaaa caatgaaaat gcgatacgaa
cacatagacc acacatttga gatccagtac aggaaagaca
cggccacgtg gaaggacagc aagaccgaga ccctccagaa
cgcccacagc atggccctgc cagccctgga gccctccacc
aggtactggg ccagggtgag ggtcaggacc tcccgcaccg
gctacaacgg gatctggagc gagtggagtg aggcgcgctc
ctgggacacc gagtcggtgc tgcctatgtg ggtgctggcc
ctcatcgtga tcttcctcac catcgctgtg ctcctggccc
tccgcttctg tggcatctac gggtacaggc tgcgcagaaa
gtgggaggag aagatcccca accccagcaa gagccacctg
ttccagaacg ggagcgcaga gctttggccc ccaggcagca
tgtcggcctt cactagcggg agtcccccac accaggggcc
gtggggcagc cgcttccctg agctggaggg ggtgttccct
gtaggattcg gggacagcga ggtgtcacct ctcaccatag
aggaccccaa gcatgtctgt gatccaccat ctgggcctga
cacgactcca gctgcctcag atctacccac agagcagccc
cccagccccc agccaggccc gcctgccgcc tcccacacac
ctgagaaaca ggcttccagc tttgacttca atgggcccta
cctggggccg ccccacagcc gctccctacc tgacatcctg
ggccagccgg agcccccaca ggagggtggg agccagaagt
ccccacctcc agggtccctg gagtacctgt gtctgcctgc
tggggggcag gtgcaactgg tccctctggc ccaggcgatg
ggaccaggac aggccgtgga agtggagaga aggccgagcc
agggggctgc agggagtccc tccctggagt ccgggggagg
ccctgcccct cctgctcttg ggccaagggt gggaggacag
gaccaaaagg acagccctgt ggctataccc atgagctctg
gggacactga ggaccctgga gtggcctctg gttatgtctc
ctctgcagac ctggtattca ccccaaactc aggggcctcg
tctgtctccc tagttccctc tctgggcctc ccctcagacc
agacccccag cttatgtcct gggctggcca gtggaccccc
tggagcccca ggccctgtga agtcagggtt tgagggctat
gtggagctcc ctccaattga gggccggtcc cccaggtcac
caaggaacaa tcctgtcccc cctgaggcca aaagccctgt
cctgaaccca ggggaacgcc cggcagatgt gtccccaaca
tccccacagc ccgagggcct ccttgtcctg cagcaagtgg
gcgactattg cttcctcccc ggcctggggc ccggccctct
ctcgctccgg agtaaacctt cttccccggg acccggtcct
gagatcaaga acctagacca ggcttttcaa gtcaagaagc
ccccaggcca ggctgtgccc caggtgcccg tcattcagct
cttcaaagcc ctgaagcagc aggactacct gtctctgccc
ccttgggagg tcaacaagcc tggggaggtg tgttgagacc
cccaggccta gacaggcaag gggatggaga gggcttgcct
tccctcccgc ctgaccttcc tcagtcattt ctgcaaagcc
aaggggcagc ctcctgtcaa ggtagctaga ggcctgggaa
aggagatagc cttgctccgg cccccttgac cttcagcaaa
tcacttctct ccctgcgctc acacagacac acacacacac
acgtacatgc acacattttt cctgtcaggt taacttattt
gtaggttctg cattattaga actttctaga tatactcatt
ccatctcccc ctcatttttt taatcaggtt tccttgcttt
tgccattttt cttccttctt ttttcactga tttattatga
gagtggggct gaggtctgag ctgagcctta tcagactgag
atgcggctgg ttgtgttgag gacttgtgtg ggctgcctgt
ccccggcagt cgctgatgca catgacatga ttctcatctg
ggtgcagagg tgggaggcac caggtgggca cccgtggggg
ttagggcttg gaagagtggc acaggactgg gcacgctcag
tgaggctcag ggaattcaga ctagcctcga ttgtcactcc
gagaaatggg catggtattg ggggtcgggg gggcggtgca
agggacgcac atgagagact gtttgggagc ttctggggag
ccctgctagt tgtctcagtg atgtctgtgg gacctccagt
cccttgagac cccacgtcat gtagagaagt taacggccca
agtggtgggc aggctggcgg gacctgggga acatcaggag
aggagtccag agcccacgtc tactgcggaa aagtcagggg
aaactgccaa acaaaggaaa atgccccaaa ggcatatatg
ctttagggcc tttggtccaa atggcccggg tggccactct
tccagataga ccaggcaact ctccctccca ccggccacag
atgaggggct gctgatctat gcctgggcct gcaccaggga
ttatggttct tttaaatctt tgcctttcag atacaggaaa
aataatggca ttaaattgct ttaatttgca ttattttagt
tatccagttt gcacatattt ttataggtat cttaggcatc
gattggtatt ttttaactgg gccaagccca ttaaggtctt
tcttctgttg ggtgctatca ttttctgatt aagtcttttt
gactattgac atacagtctt tcacagatgg tggagtgttt
ttcccccaaa tctgttgttt gtcttataat gttgtatatg
aggttttatg gtgtatgaat atgaatgctt ctgtaatgtc
aaacagatcc ctagtaaact ccttcttcac ttttactgtc
agatttacaa aggtcctccc attgcaaagc agtgtttgtc
ctaatttata tattgttttt ctagttcatt ttgtgtttcc
aacttttcat gtaaaatttt aattattttt gaatgtgtgg
atgtgagact gaggtgcctt ttggtactga aattcttttt
ccatgtacct gaagtgttac ttttgtgata taggaaatcc
ttgtatatat actttattgg tccctaggct tcctattttg
ttaccttgct ttctctatgg catccaccat tttgattgtt
ctacttttat gatatgtttt cataagtggt taagcaagta
ttctcgttac ttttgctctt aaatccctat tcattacagc
aatgttggtg gtcaaagaaa atgataaaca acttgaatgt
tcaatggtcc tgaaatacat aacaacattt tagtacattg
taaagtagaa tcctctgttc ataatgaaca agatgaacca
atgtggatta gaaagaagtc cgagatatta attccaaaat
atccagacat tgttaaaggg aaaaaattgc aataaaatat
ttgtaacata aaa

(SEQ ID NO: 6)

MVLAQGLLSM ALLALCWERS LAGAEETIPL QTLRCYNDYT
SHITCRWADT QDAQRLVNVT LIRRVNEDLL EPVSCDLSDD
MPWSACPHPR CVPRRCVIPC QSFVVTDVDY FSFQPDRPLG
TRLTVTLTQH VQPPEPRDLQ ISTDQDHELL TWSVALGSPQ
SHWLSPGDLE FEVVYKRLQD SWEDAAILLS NTSQATLGPE
HLMPSSTYVA RVRTRLAPGS RLSGRPSKWS PEVCWDSQPG
DEAQPQNLEC FFDGAAVLSC SWEVRKEVAS SVSFGLFYKP
SPDAGEEECS PVLREGLGSL HTRHHCQIPV PDPATHGQYI
VSVQPRRAEK HIKSSVNIQM APPSLNVIKD GDSYSLRWET
MKMRYEHIDH TFEIQYRKDT ATWKDSKTET LQNAHSMALP
ALEPSTRYWA RVRVRTSRTG YNGIWSEWSE ARSWDTESVL
PMWVLALIVI FLTIAVLLAL RFCGIYGYRL RRKWEEKIPN
PSKSHLFQNG SAELWPPGSM SAFTSGSPPH QGPWGSRFPE
LEGVFPVGFG DSEVSPLTIE DPKHVCDPPS GPDTTPAASD
LPTEQPPSPQ PGPPAASHTP EKQASSFDFN GPYLGPPHSR
SLPDILGQPE PPQEGGSQKS PPPGSLEYLC LPAGGQVQLV
PLAQAMGPGQ AVEVERRPSQ GAAGSPSLES GGGPAPPALG
PRVGGQDQKD SPVAIPMSSG DTEDPGVASG YVSSADLVET
PNSGASSVSL VPSLGLPSDQ TPSLCPGLAS GPPGAPGPVK
SGFEGYVELP PIEGRSPRSP RNNPVPPEAK SPVLNPGERP
ADVSPTSPQP EGLLVLQQVG DYCFLPGLGP GPLSLRSKPS
SPGPGPEIKN LDQAFQVKKP PGQAVPQVPV IQLFKALKQQ
DYLSLPPWEV NKPGEVC

The protein encoded by this gene is the common beta chain of the high affinity receptor for IL-3, IL-5 and CSF. Defects in this gene have been reported to be associated with protein alveolar proteinosis (PAP).

### PVALB

In some embodiments, the Chromosome 15 QTL comprises an PVALB gene. Thus, some aspects of the present disclosure provide methods that comprise administering to a subject (e.g., a cancer patient) an agent that modifies expression of the human Parvalbumin (PVALB) gene or modifies the function of the gene product. The PVALB gene is located on human Chromosome 22 (36,800,684-36,819,479; GRCh38: CM000684.2).

The protein encoded by this gene is a high affinity calcium ion-binding protein that is structurally and functionally similar to calmodulin and troponin C. The encoded protein is thought to be involved in muscle relaxation. Alternative splicing results in multiple transcript variants. Below is a non-limiting example of a human PVALB gene and a human PVALB protein sequence:

(SEQ ID NO: 7)

acttgctctg cccgctcaaa cagttgcagg atgtcgatga
cagacttgct gaacgctgag gacatcaaga aggcggtggg
agcctttagc gctaccgact ccttcgacca caaaaagttc
ttccaaatgg tcggcctgaa gaaaaagagt gcggatgatg
tgaagaaggt gtttcacatg ctggacaagg acaaaagtgg
cttcatcgag gaggatgagc tgggattcat cctaaaaggc
ttctccccag atgccagaga cctgtctgct aaagaaacca
agatgctgat ggctgctgga gacaaagatg gggacggcaa
aattggggtt gacgaattct ccactctggt ggctgaaagc
taagaagcac tgactgcccc tggtcttcca cctctctgcc
ctgaacaccc aatctcggcc cctctcgcca ccctcctgca
tttctgttca gttcgtttat gttatttttt actcccccat
cccctgtggc cctctaatga caccattctt ctggaaaatg
ctggagaagc aataaaggtt gtaccagtca

(SEQ ID NO: 8)

MSMTDLLNAE DIKKAVGAFS ATDSFDHKKF FQMVGLKKKS
ADDVKKVFHM LDKDKSGFIE EDELGFILKG FSPDARDLSA
KETKMLMAAG DKDGDGKIGV DEFSTLVAES

### Therapeutic Agents

The present disclosure contemplates various gene and protein targets for an agent that modifies expression of a gene or function of a product of the gene. For example, the present disclosure contemplates human NCF4 as a gene target and/or human NCF4 as a protein target for modulating immune checkpoint inhibitor response in subjects (e.g., cancer patients). Thus, an agent administered to a subject (e.g., a cancer patient) may be an agent that modifies expression of a gene or function of a product (e.g., mRNA or protein) of the gene. The agent may be selected from, for example, antibodies, small molecule drug, and gene editing molecules.

### Antibodies

In some embodiments, an agent that modifies function of a protein encoded by a gene target of the present disclosure is an antibody. In some embodiments, the antibody binds specifically to human NCF4 (e.g., a monoclonal human or humanized anti-NCF4 antibody) or a protein downstream from the NCF4 cellular pathway. In other embodiments, the antibody binds specifically to human p40-phox (e.g., a monoclonal human or humanized anti-p40-phox antibody) or another protein that forms the nicotinamide adenine dinucleotide phosphate (NADPH) oxidase enzyme complex. In some embodiments, the antibody binds to a protein in the reactive oxygen species (ROS) pathway.

The term “antibody” encompasses antibodies or immunoglobulins of any isotype, including but not limited to humanized antibodies and chimeric antibodies. An antibody may be a single-chain antibody (scAb) or a single domain antibody (dAb) (e.g., a single domain heavy chain antibody or a single domain light chain antibody; see Holt et al. (2003) Trends Biotechnol. 21:484). The term “antibody” also encompasses fragments of antibodies (antibody fragments) that retain specific binding to an antigen. “Antibody” further includes single-chain variable fragments (scFvs), which are fusion proteins of the variable regions of the heavy (VH) and light chains (VL) of antibodies, connected with a short linker peptide, and diabodies, which are noncovalent dimers of scFv fragments that include the VH and VL connected by a small peptide linker (Zapata et al., Protein Eng. 8 (10): 1057-1062 (1995)). Other fusion proteins that comprise an antigen-binding portion of an antibody and a non-antibody protein are also encompassed by the term “antibody.”

Antibody fragments comprise a portion of an intact antibody, for example, the antigen binding or variable region of the intact antibody. Examples of antibody fragments include an antigen-binding fragment (Fab), Fab′, F(ab′)2, a variable domain Fv fragment (Fv), an Fd fragment, and an antigen binding fragment of a chimeric antigen receptor.

Papain digestion of antibodies produces two identical antigen-binding fragments, referred to as “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen.

Fv is the minimum antibody fragment that contains a complete antigen-recognition and -binding site. This region includes a dimer of one heavy-chain variable domain and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

Fab fragments contain the constant domain of the light chain and the first constant domain (CH1) of the heavy chain. Fab fragments differ from Fab′ fragments by the addition of a few residues at the carboxyl terminus of the heavy chain CH1 domain including at least one cysteine from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

scFv antibody fragments comprise the VH and VL of an antibody, wherein these regions are present in a single polypeptide chain. In some cases, the Fv polypeptide further comprises a polypeptide linker between the VH and VL regions, which enables the scFv to form the desired structure for antigen binding. For a review of scFv, see Pluckthun in The Pharmacology of Monoclonal Antibodies, vol. 113, Rosenburg and Moore eds., Springer-Verlag, New York, pp. 269-315 (1994).

Diabody refers to a small antibody fragment with two antigen-binding sites, which fragments comprise a VH connected to a VL in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, Hollinger et al. Proc. Natl. Acad. Sci. USA 90:6444-6448 (1993).

An antibody can be monovalent or bivalent. An antibody can be an Ig monomer, which is a “Y-shaped” molecule that consists of four polypeptide chains: two heavy chains and two light chains connected by disulfide bonds.

Antibodies can be detectably labeled, e.g., with a radioisotope, an enzyme that generates a detectable product, and/or a fluorescent protein. Antibodies can be further conjugated to other moieties, such as members of specific binding pairs, e.g., biotin member of biotin-avidin specific binding pair. Antibodies can also be bound to a solid support, including, but not limited to, polystyrene plates and/or beads.

An isolated antibody is one that has been identified and separated and/or recovered from a component of its natural environment (i.e., is not naturally occurring). Contaminant components of its natural environment are materials that would interfere with uses (e.g., diagnostic or therapeutic uses) of the antibody, and can include enzymes, hormones, and other proteinaceous or nonproteinaceous solutes. In some cases, an antibody is purified (1) to greater than 90%, greater than 95%, or greater than 98% by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or non-reducing conditions using Coomassie blue or silver stain. Isolated antibodies encompass antibodies in situ within recombinant cells, as at least one component of the antibody's natural environment will not be present. In some embodiments, an isolated antibody is prepared by at least one purification step.

A monoclonal antibody is an antibody produced by a group of identical cells, all of which were produced from a single cell by repetitive cellular replication. That is, the clone of cells only produces a single antibody species. While a monoclonal antibody can be produced using hybridoma production technology, other production methods known to those skilled in the art can also be used (e.g., antibodies derived from antibody phage display libraries).

A complementarity determining region (CDR) is the non-contiguous antigen combining sites found within the variable region of both heavy and light chain polypeptides. CDRs have been described by Lefranc et al. (2003) Developmental and Comparative Immunology 27:55; Kabat et al., J. Biol. Chem. 252:6609-6616 (1977); Kabat et al., U. S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein.

As used herein, the terms “CDR-L1,” “CDR-L2,” and “CDR-L3” refer, respectively, to the first, second, and third CDRs in a light chain variable region. As used herein, the terms “CDR-H1”, “CDR-H2”, and “CDR-H3” refer, respectively, to the first, second, and third CDRs in a heavy chain variable region. As used herein, the terms “CDR-1”, “CDR-2”, and “CDR-3” refer, respectively, to the first, second and third CDRs of either chain's variable region.

A framework when used in reference to an antibody variable region includes all amino acid residues outside the CDR regions within the variable region of an antibody. A variable region framework is generally a discontinuous amino acid sequence that includes only those amino acids outside of the CDRs. A “framework region” includes each domain of the framework that is separated by the CDRs.

A humanized antibody is an antibody comprising portions of antibodies of different origin, wherein at least one portion comprises amino acid sequences of human origin. For example, the humanized antibody can comprise portions derived from an antibody of nonhuman origin with the requisite specificity, such as a mouse, and from antibody sequences of human origin (e.g., chimeric immunoglobulin), joined together chemically by conventional techniques (e.g., synthetic) or prepared as a contiguous polypeptide using genetic engineering techniques (e.g., DNA encoding the protein portions of the chimeric antibody can be expressed to produce a contiguous polypeptide chain). Another example of a humanized antibody is an antibody containing at least one chain comprising a CDR derived from an antibody of nonhuman origin and a framework region derived from a light and/or heavy chain of human origin (e.g., CDR-grafted antibodies with or without framework changes). Chimeric or CDR-grafted single chain antibodies are also encompassed by the term humanized immunoglobulin. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Padlan, E. A. et al., European Patent Application No. 0,519,596 A1. See also, Ladner et al., U.S. Pat. No. 4,946,778; Huston, U.S. Pat. No. 5,476,786; and Bird, R. E. et al., Science, 242:423-426 (1988)), regarding single chain antibodies.

In some embodiments, a humanized antibody is produced using synthetic and/or recombinant nucleic acids to prepare genes (e.g., cDNA) encoding the desired humanized chain. For example, nucleic acid (e.g., DNA) sequences coding for humanized variable regions can be constructed using PCR mutagenesis methods to alter DNA sequences encoding a human or humanized chain, such as a DNA template from a previously humanized variable region (see e.g., Kamman, M., et al., Nucl. Acids Res., 17:5404 (1989)); Sato, K., et al., Cancer Research, 53:851-856 (1993); Daugherty, B. L. et al., Nucleic Acids Res., 19 (9): 2471-2476 (1991); and Lewis, A. P. and J. S. Crowe, Gene, 101:297-302 (1991)). Using these or other suitable methods, variants can also be readily produced. For example, cloned variable regions can be mutagenized, and sequences encoding variants with the desired specificity can be selected (e.g., from a phage library; see e.g., Krebber et al., U.S. Pat. No. 5,514,548; Hoogenboom et al., WO 93/06213, published Apr. 1, 1993).

### Small Molecules

In some embodiments, an agent that modifies function of a protein encoded by a gene target of the present disclosure is a small molecule drug, for example, a small molecule inhibitor. In some embodiments, the agent is a small molecule inhibitor of human NCF4.

Small molecule drugs are chemical compounds with a molecular weight in the range of 0.1-1 kDa. They are smaller than biologics or bio-therapeutic modalities, which are generally more than 1 kDa in molecular size. Owing to the small size, small molecule drugs possess an advantage over biologics to target not only the extracellular components like cell surface receptors or protein domains attached to the cell membranes like glycoproteins but also the intracellular proteins like different kinases, as they can easily cross the outer plasma membrane of the cell. They are easy to synthesize by chemical reactions and are cheaper than biologics (Buvailo, 2018). They are mostly taken orally by the patients and are designed to be metabolized from an inactive prodrug to an active compound. The small-molecule drugs are developed to follow Lipinski's rule of five to be made bioavailable to the patient and be cleared from the body after its action. The Lipinski's rule of five-ADME governs that small-molecule drug has properties to be adsorbed (A) by the human body, be easily distributed (D) inside the human body, metabolized (M) to an active drug, and then later excreted (E) out form the system (Lipinski, 2004). Most of the therapeutic drugs (˜90%) generated by pharma industries are still small molecules and cannot wholly be replaced by biologics in future (Buvailo, 2018; Cohen, 2015).

### Gene Editing

In some embodiments, gene editing is used to modify expression of a gene target of the present disclosure. Gene editing methods include, without limitation, the use of programmable nucleases or the use of RNA interference (RNAi). Antisense oligonucleotides (ASOs) are also contemplated herein. In some embodiments, the gene target is human NCF4.

### Programmable Nucleases

In some embodiments, an agent used to modify expression of a gene target (e.g., NCF4) is a programmable nuclease. Non-limiting examples of programmable nuclease-based systems include clustered regularly interspaced short palindromic repeat (CRISPR) systems, zinc-finger nucleases (ZFNs), and transcription activator-like effector nucleases (TALENs). See, e.g., Carroll D Genetics. 2011; 188 (4): 773-782; Joung J K et al. Nat Rev Mol Cell Biol. 2013; 14 (1): 49-55; and Gaj T et al. Trends Biotechnol. 2013 July; 31 (7): 397-405, each of which is incorporated by reference herein.

The CRISPR/Cas system is a naturally occurring defense mechanism in prokaryotes that has been repurposed as an RNA-guided-DNA-targeting platform for gene editing. Engineered CRISPR systems contain two main components: a guide RNA (gRNA) and a CRISPR-associated endonuclease (e.g., Cas protein). The gRNA is a short synthetic RNA composed of a scaffold sequence for nuclease-binding and a user-defined nucleotide spacer (e.g., ˜15-25 nucleotides, or ˜20 nucleotides) that defines the genomic target (e.g., gene) to be modified. Thus, one can change the genomic target of the Cas protein by simply changing the target sequence present in the gRNA. In some embodiments, the Cas9 endonuclease is from Streptococcus pyogenes (NGG PAM) or Staphylococcus aureus (NNGRRT or NNGRR (N) PAM), although other Cas9 homologs, orthologs, and/or variants (e.g., evolved versions of Cas9) may be used, as provided herein. Additional non-limiting examples of RNA-guided nucleases that may be used as provided herein include Cpf1 (TTN PAM); SpCas9 D1135E variant (NGG (reduced NAG binding) PAM); SpCas9 VRER variant (NGCG PAM); SpCas9 EQR variant (NGAG PAM); SpCas9 VQR variant (NGAN or NGNG PAM); Neisseria meningitidis (NM) Cas9 (NNNNGATT PAM); Streptococcus thermophilus (ST) Cas9 (NNAGAAW PAM); and Treponema denticola (TD) Cas9 (NAAAAC). In some embodiments, the CRISPR-associated endonuclease is selected from Cas9, Cas12a/Cpf1, C2c1, and C2c3. In some embodiments, the Cas nuclease is Cas9. In some embodiments, the Cas nuclease is Cas12a.

A guide RNA comprises at least a spacer sequence that hybridizes to (binds to) a target nucleic acid sequence and a CRISPR repeat sequence that binds the endonuclease and guides the endonuclease to the target nucleic acid sequence. As is understood by the person of ordinary skill in the art, each gRNA is designed to include a spacer sequence complementary to its genomic target sequence. See, e.g., Jinek et al., Science, 2012; 337:816-821 and Deltcheva et al., Nature, 2011; 471:602-607, each of which is incorporated by reference herein.

In some embodiments, a gRNA comprises a spacer sequence complementary to a human homolog (e.g., human ortholog) of a gene that maps to a mouse Chr 15 QTL, e.g., GRCm38/mm10 Chr15: 75.79-81.31 Mb, such as GRCm38/mm10 Chr15: 78,175,584-78,274,852 Mb or GRCm38/mm10 Chr15: 79,998,008-80,088,247 Mb. In some embodiments, a gRNA comprises a spacer sequence complementary to a human NCF4 gene. In some embodiments, a gRNA comprises a spacer sequence complementary to a human NCF4 gene variant, e.g., a variant comprising a SNP encoding human NCF4 with a T85N substitution. In some embodiments, a gRNA comprises a spacer sequence complementary to a gene located within 2 Mb upstream and/or downstream from the human NCF4 gene. In some embodiments, a gRNA comprises a spacer sequence complementary to a sequence of SEQ ID NO: 1 or the reverse complement of the sequence of SEQ ID NO: 1.

The concentration of CRISPR-associated endonuclease or nucleic acid encoding a CRISPR-associated endonuclease may vary. In some embodiments, the concentration is 100 ng/μl to 1000 ng/μl. For example, the concentration may be 100, 150, 200, 250, 300, 350, 400, 450, 500, 550, 600, 650, 700, 750, 800, 850, 900, 950, or 1000 ng/μl. In some embodiments, the concentration is 100 ng/μl to 500 ng/μl, or 200 ng/μl to 500 ng/μl.

The concentration of gRNA may also vary. In some embodiments, the concentration is 200 ng/μl to 2000 ng/μl. For example, the concentration may be 200, 300, 400, 500, 600, 700, 800, 900, 1000, 1100, 1200, 1300, 1400, 1500, 1600, 1700, 1700, 1900, or 2000 ng/μl.

In some embodiments, the concentration is 500 ng/μl to 1000 ng/μl. In some embodiments, the concentration is 100 ng/μl to 1000 ng/μl. For example, the concentration may be 100, 150, 200, 250, 300, 350, 400, 450, 500, 550, 600, 650, 700, 750, 800, 850, 900, 950, or 1000 ng/μl.

In some embodiments, the ratio of concentration of CRISPR-associated endonuclease or nucleic acid encoding the CRISPR-associated endonuclease to the concentration of gRNA is 2:1. In other embodiments, the ratio of concentration of CRISPR-associated endonuclease or nucleic acid encoding the CRISPR-associated endonuclease to the concentration of gRNA is 1:1.

A donor nucleic acid typically includes a sequence of interest flanked by homology arms. Homology arms are regions of the ssDNA that are homologous to regions of genomic DNA located in a genomic locus. One homology arm is located to the left (5′) of a genomic region of interest (into which a sequence of interest is introduced) (the left homology arm) and another homology arm is located to the right (3′) of the genomic region of interest (the right homology arm). These homology arms enable homologous recombination between the ssDNA donor and the genomic locus, resulting in insertion of the sequence of interest into the genomic locus of interest (e.g., via CRISPR/Cas9-mediated homology directed repair (HDR)).

The homology arms may vary in length. For example, each homology arm (the left arm and the right homology arm) may have a length of 20 nucleotide bases to 1000 nucleotide bases. In some embodiments, each homology arm has a length of 20 to 200, 20 to 300, 20 to 400, 20 to 500, 20 to 600, 20 to 700, 20 to 800, or 20 to 900 nucleotide bases. In some embodiments, each homology arm has a length of 20, 30, 40, 50, 60, 70, 80, 90, 100, 150, 200, 250, 300, 350, 400, 450, 500, 550, 600, 650, 700, 750, 800, 850, 900, 950, or 1000 nucleotide bases. In some embodiments, the length of one homology arm differs from the length of the other homology arm. For example, one homology arm may have a length of 20 nucleotide bases, and the other homology arm may have a length of 50 nucleotide bases. In some embodiments, the donor DNA is single stranded. In some embodiments, the donor DNA is double stranded. In some embodiments, the donor DNA is modified, e.g., via phosphorothioation. Other modifications may be made.

In some embodiments, the programmable nuclease is a zinc finger nucleases (ZFNs). ZFNs are typically fusion proteins that include a DNA-binding domain derived from a zinc-finger protein linked to a cleavage domain. The most common cleavage domain is the Type IIS enzyme Fok1. Fok1 catalyzes double-stranded cleavage of DNA, at 9 nucleotides from its recognition site on one strand and β nucleotides from its recognition site on the other. See, for example, U.S. Pat. Nos. 5,356,802; 5,436,150 and 5,487,994; as well as Li et al. Proc., Natl. Acad. Sci. USA 89 (1992): 4275-4279; Li et al. Proc. Natl. Acad. Sci. USA, 90:2764-2768 (1993); Kim et al. Proc. Natl. Acad. Sci. USA. 91:883-887 (1994a); Kim et al. J. Biol. Chem. 269:31,978-31,982 (1994b). One or more of these enzymes (or enzymatically functional fragments thereof) can be used as a source of cleavage domains.

The DNA-binding domain, which can, in principle, be designed to target any genomic location of interest, can be a tandem array of Cys2His2 zinc fingers, each of which generally recognizes three to four nucleotides in the target DNA sequence. The Cys2His2 domain has a general structure: Phe (sometimes Tyr)-Cys-(2 to 4 amino acids)-Cys-(3 amino acids)-Phe (sometimes Tyr)-(5 amino acids)-Leu-(2 amino acids)-His-(3 amino acids)-His. By linking together multiple fingers (the number varies: three to six fingers have been used per monomer in published studies), ZFN pairs can be designed to bind to genomic sequences 18-36 nucleotides long.

In some embodiments, the programmable nuclease is a transcription activator-like effector nuclease (TALEN). TALENs have an overall architecture similar to that of ZFNs, with the main difference that the DNA-binding domain comes from TAL effector proteins, transcription factors from plant pathogenic bacteria. The DNA-binding domain of a TALEN is a tandem array of amino acid repeats, each about 34 residues long. The repeats are very similar to each other; typically, they differ principally at two positions (amino acids 12 and 13, called the repeat variable diresidue, or RVD). Each RVD specifies preferential binding to one of the four possible nucleotides, meaning that each TALEN repeat binds to a single base pair, though the NN RVD is known to bind adenines in addition to guanine. TAL effector DNA binding is mechanistically less well understood than that of zinc-finger proteins, but their seemingly simpler code could prove very beneficial for engineered-nuclease design. TALENs also cleave as dimers, have relatively long target sequences (the shortest reported so far binds 13 nucleotides per monomer) and appear to have less stringent requirements than ZFNs for the length of the spacer between binding sites. Monomeric and dimeric TALENs can include more than 10, more than 14, more than 20, or more than 24 repeats.

Methods of engineering TAL to bind to specific nucleic acids are described in Cermak, et al, Nucl. Acids Res. 1-11 (2011). U.S. Published Application No. 2011/0145940, which discloses TAL effectors and methods of using them to modify DNA. Miller et al. Nature Biotechnol 29:143 (2011) reported making TALENs for site-specific nuclease architecture by linking TAL truncation variants to the catalytic domain of Fok1 nuclease. The resulting TALENs were shown to induce gene modification in immortalized human cells. General design principles for TALE binding domains can be found in, for example, WO 2011/072246

### RNA Interference

In some embodiments, an agent used to modify expression of a gene target (e.g., NCF4) is an RNA interference (RNAi) molecule. Non-limiting examples of RNAi molecules include small interfering RNA (siRNA), micro-RNA (miRNA) and short hairpin RNA (shRNA). RNAi is a gene-silencing process that targets mRNA hence lowering protein expression. The mechanism of RNAi is based on the sequence-specific degradation of host mRNA through the cytoplasmic delivery of double-stranded RNA (dsRNA) identical to the target sequence. Degradation of target gene expression is achieved through an enzymatic pathway involving the endogenous RNA-induced silencing complex (RISC). One strand of the siRNA duplex (the guide strand) is loaded into the RISC with the assistance of Argonaute (Ago) proteins and double-stranded RNA-binding proteins. The RISC then localizes the guide strand to the complementary mRNA molecule, which is subsequently cleaved by Ago near the middle of the hybrid. The cleaved mRNA is further degraded by other endogenous nucleases. Likewise, the RISC also plays an important cellular role in inhibiting endogenously derived mRNA through a related miRNA mechanism.

Several methods of RNAi have evolved over time, with the simplest approach involving the transfection of chemically synthesized short interfering RNA oligonucleotides (siRNAs) directly into the cytosol. While the delivery of siRNAs can be achieved in many cell types, variable transfection efficiencies have limited siRNA-mediated RNAi to only those cells capable of transfection. Another form of RNAi involves the use of short hairpin RNAs (shRNAs) synthesized within the cell by DNA vector-mediated production. Like siRNAs, shRNAs may be transfected as plasmid vectors encoding shRNAs transcribed by RNA pol III or modified pol II promoters, but can also be delivered into mammalian cells through infection of the cell with virally produced vectors. While siRNA delivers the siRNA duplex directly to the cytosol, shRNAs are capable of DNA integration and consist of two complementary 19-22 bp RNA sequences linked by a short loop of 4-11 nt similar to the hairpin found in naturally occurring miRNA. Following transcription, the shRNA sequence is exported to the cytosol where it is recognized by an endogenous enzyme, Dicer, which processes the shRNA into the siRNA duplexes. Like the exogenously delivered synthetic siRNA oligonucleotides, this endogenously derived siRNA binds to the target mRNA and is incorporated into the RISC complex for target-specific mRNA degradation.

Although siRNA and shRNA ultimately utilize a similar cellular mechanism (RISC), the choice of which method to use depends on several factors such as cell type, time demands, and the need for transient versus stable integration. There are a variety of reagents available for siRNA design and synthesis. Therefore, the efficiency of knockdown for each siRNA sequence can be rapidly determined and, in fact, there are several commercial sources for siRNA which have been functionally validated. In addition, siRNA delivery has benefited from the plethora of transfection reagents already in existence, yielding a potentially high level of gene silencing with minimal cellular toxicity.

The proper selection of a target sequence for a given gene of interest (e.g., NCF4) is an important component of successful gene knockdown regardless of the RNAi methodology. Although target RNAi sequences have been constructed from 19 to 27 bp, most data on effective sequence selection involve the design of 19 bp targets. Numerous algorithms have been designed to predict these 19 bp targets with a nucleotide composition thought to confer the highest efficacy (Ui-Tei K et al. Nucl Acids Res. 2004; 32:936-948; Taxman D J et al. BMC Biotechnol. 2006; 6:7; Reynolds A et al. Nat Biotechnol. 2004; 22:326-330; and Amarzguioui M et al. Biochem Biophys Res Commun. 2004; 316:1050-1058, each of which is incorporated herein by reference). See also Moore C B et al. Methods Mol Biol. 2010; 629:141-158, incorporated herein by reference.

In some embodiments, an RNAi molecule comprises sequence complementary to a human homolog (e.g., human ortholog) of a gene that maps to a mouse Chr 15 QTL, e.g., GRCm38/mm10 Chr15: 75.79-81.31 Mb, such as GRCm38/mm10 Chr15: 78,175,584-78,274,852 Mb or GRCm38/mm10 Chr15: 79,998,008-80,088,247 Mb. In some embodiments, an RNAi molecule comprises sequence complementary to a human NCF4 gene. In some embodiments, an RNAi molecule comprises sequence complementary to a human NCF4 gene variant, e.g., a variant comprising a SNP encoding human NCF4 with a T85N substitution. In some embodiments, an RNAi molecule comprises sequence complementary to a gene located within 2 Mb upstream and/or downstream from the human NCF4 gene. In some embodiments, an RNAi molecule comprises sequence complementary to a sequence of SEQ ID NO: 1 or the reverse complement of the sequence of SEQ ID NO: 1.

### Antisense Oligonucleotides

In some embodiments, an agent used to modify expression of a gene target (e.g., NCF4) is an antisense oligonucleotide (ASO). An ASO is a single-stranded deoxyribonucleotide, which is complementary to the mRNA target (e.g., NCF4 mRNA). The goal of the antisense approach is the downregulation of a molecular target, usually achieved by induction of RNase H endonuclease activity that cleaves the RNA-DNA heteroduplex with a significant reduction of the target gene translation. Other ASO-driven mechanisms include inhibition of 5′ cap formation, alteration of splicing process (splice-switching), and steric hindrance of ribosomal activity. See, e.g., Di Fusco D et al. Front. Pharmacol., 29 Mar. 2019, incorporated herein by reference.

In some embodiments, an ASO comprises sequence complementary to a human homolog (e.g., human ortholog) of a gene that maps to a mouse Chr 15 QTL, e.g., GRCm38/mm10 Chr15: 75.79-81.31 Mb, such as GRCm38/mm10 Chr15: 78,175,584-78,274,852 Mb or GRCm38/mm10 Chr15: 79,998,008-80,088,247 Mb. In some embodiments, an ASO comprises sequence complementary to a human NCF4 gene. In some embodiments, an ASO comprises sequence complementary to a human NCF4 gene variant, e.g., a variant comprising a SNP encoding human NCF4 with a T85N substitution. In some embodiments, an ASO comprises sequence complementary to a gene located within 2 Mb upstream and/or downstream from the human NCF4 gene. In some embodiments, an ASO comprises sequence complementary to a sequence of SEQ ID NO: 1 or the reverse complement of the sequence of SEQ ID NO: 1.

### Immune Checkpoint Inhibitors

The present disclosure provides, in some aspects, methods comprising administering to a subject (e.g., a cancer patient) an immune checkpoint inhibitor. Immune checkpoints regulate T cell function in the immune system. T cells play a central role in cell-mediated immunity. Checkpoint proteins interact with specific ligands which send a signal into the T cell and essentially switch off or inhibit T cell function. Cancer cells take advantage of this system by driving high levels of expression of checkpoint proteins on their surface which results in control of the T cells expressing checkpoint proteins on the surface of T cells that enter the tumor microenvironment, thus suppressing the anticancer immune response. As such, inhibition of checkpoint proteins would result in restoration of T cell function and an immune response to the cancer cells. Examples of checkpoint proteins include, but are not limited to CTLA-4, PDL1, PDL2, PD-1, B7-H3, B7-H4, BTLA, HVEM, TIM3, GAL9, LAG3, VISTA, KIR, 2B4 (belongs to the CD2 family of molecules and is expressed on all NK, γδ, and memory CD8+ (αβ) T cells), CD160 (also referred to as BY55), CGEN-15049, CHK 1 and CHK2 kinases, A2aR and various B-7 family ligands.

PD-1 is a checkpoint protein on immune cells called T cells. It normally acts as a type of “off switch” that helps keep the T cells from attacking other cells in the body. It does this when it attaches to PD-L1, a protein on some normal (and cancer) cells. When PD-1 binds to PD-L1, it basically tells the T cell to leave the other cell alone. Some cancer cells have large amounts of PD-L1, which helps them hide from an immune attack. Monoclonal antibodies that target either PD-1 or PD-L1 can block this binding and boost the immune response against cancer cells. These drugs have shown a great deal of promise in treating certain cancers. In some embodiments, the immune checkpoint inhibitor is a PD-1 inhibitor, for example, an anti-PD-1 antibody. In some embodiments, the immune checkpoint inhibitor is a PD-L1 inhibitor, for example, an anti-PD-L1 antibody. Non-limiting examples of anti-PD-1 antibodies include pembrolizumab, nivolumab, dostarlimab, and cemiplimab. Non-limiting examples of anti-PD-L1 antibodies include atezolizumab, durvalumab, and avelumab. Cancers associated with anti-PD-1 antibodies and anti-PD-L1 antibodies include, for example, basal cell carcinoma, bladder cancer, breast cancer, cervical cancer, colorectal cancer, endometrial cancer, esophageal carcinoma, gastric cancer, head and neck cancer, hepatocellular carcinoma, hodgkin's lymphoma, malignant pleural mesothelioma, melanoma, melanoma, metastatic, merkel cell carcinoma, non-small cell lung cancer, primary mediastinal large b-cell lymphoma, renal cell carcinoma, small cell lung cancer, solid tumors, squamous cell carcinoma, stomach cancer, and urothelial carcinoma. Thus, a cancer patient of the present disclosure may be diagnosed with any one of the preceding cancers. Other cancers are also contemplated herein.

CTLA-4 is another protein on some T cells that acts as a type of “off switch” to keep the immune system in check. In some embodiments, the immune checkpoint inhibitor is a CTLA-4 inhibitor, for example, an anti-CTLA-4 antibody. A non-limiting example of anti-CTLA-4 antibodies is ipilimumab. Cancers associated with anti-CTLA-4antibodies include, for example, colorectal cancer, malignant pleural mesothelioma, melanoma, melanoma, metastatic, renal cell carcinoma. Thus, a cancer patient of the present disclosure may be diagnosed with any one of the preceding cancers. Other cancers are also contemplated herein.

### Therapeutic Methods and Applications

The agents and/or ICIs described herein may be used in a variety of therapeutic applications, such as anti-cancer therapeutics, or for biological research.

In some embodiments, the subject may be a neonate, a juvenile, or an adult. Of particular interest are mammalian subjects. Mammalian species that may be treated with the present methods include canines and felines; equines; bovines; ovines; etc. and primates, particularly humans. Animal models, particularly small mammals (e.g. mice, rats, guinea pigs, hamsters, rabbits, etc.) may be used for experimental investigations. Administration of agents and/or ICIs described herein and compositions thereof can occur by injection, irrigation, inhalation, consumption, electro-osmosis, hemodialysis, iontophoresis, and other methods known in the art. In some embodiments, administration route is local or systemic. In some embodiments administration route is intraarterial, intracranial, intradermal, intraduodenal, intrammamary, intrameningeal, intraperitoneal, intrathecal, intratumoral, intravenous, intravitreal, ophthalmic, parenteral, spinal, subcutaneous, ureteral, urethral, vaginal, or intrauterine.

In some embodiments, the administration route is by infusion (e.g., continuous or bolus). Examples of methods for local administration, that is, delivery to the site of injury or disease, include through an Ommaya reservoir, e.g. for intrathecal delivery (See e.g., U.S. Pat. Nos. 5,222,982 and 5,385,582, incorporated herein by reference); by bolus injection, e.g. by a syringe, e.g. into a joint; by continuous infusion, e.g. by cannulation, such as with convection; or by implanting a device upon which the cells have been reversibly affixed. In some embodiments, the administration route is by topical administration or direct injection.

In some embodiments, introducing an agent and/or ICI into the subject may be a one-time event. In some embodiments, such treatment may require an on-going series of repeated treatments. In some embodiments, multiple administrations of the agent and/or ICI may be required before an effect is observed. The exact protocols depend upon the disease or condition, the stage of the disease and parameters of the individual subject being treated.

In some embodiments, the agents and/or ICIs described herein are administered to a subject in order to treat a disease, such as cancer. In some embodiments, treatment comprises delivering an effective amount of an agent and/or ICI, or composition thereof, to a subject in need thereof. In some embodiments, treating refers to the treatment of a disease in a mammal, e.g., in a human, including (a) inhibiting the disease, i.e., arresting disease development or preventing disease progression; (b) relieving the disease, i.e., causing regression of the disease state or relieving one or more symptoms of the disease; and (c) curing the disease, i.e., remission of one or more disease symptoms. In some embodiments, treatment may refer to a short-term (e.g., temporary and/or acute) and/or a long-term (e.g., sustained) reduction in one or more disease symptoms. In some embodiments, treatment results in an improvement or remediation of the symptoms of the disease. The improvement is an observable or measurable improvement, or may be an improvement in the general feeling of well-being of the subject.

The effective amount of an agent and/or ICI administered to a particular subject will depend on a variety of factors, several of which will differ from patient to patient including the disorder being treated and the severity of the disorder; activity of the specific agent(s) employed; the age, body weight, general health, sex and diet of the patient; the timing of administration, route of administration; the duration of the treatment; drugs used in combination; the judgment of the prescribing physician; and like factors known in the medical arts.

In some embodiments, the effective amount of an agent and/or ICI may be the amount required to result in at least 1, 2, 3, 4, 5, 6, 7, 8, 9, 10, 20, 30, 40, 50, 60, 70, 80, 90, 100, or more fold decrease in tumor mass or volume, decrease in the number of tumor cells, or decrease in the number of metastases. In some embodiments, the effective amount of an agent and/or ICI may be the amount required to achieve an increase in life expectancy, an increase in progression-free or disease-free survival, or amelioration of various physiological symptoms associated with the disease being treated.

In some embodiments, the agent and/or ICI described herein may be used in the treatment of a cell-proliferative disorder, such as a cancer. Cancers that may be treated using the compositions and methods disclosed herein include cancers of the blood and solid tumors. For example, cancers that may be treated using the compositions and methods disclosed herein include, but are not limited to, adenoma, carcinoma, sarcoma, leukemia or lymphoma. In some embodiments, the cancer is chronic lymphocytic leukemia (CLL), B cell acute lymphocytic leukemia (B-ALL), acute lymphoblastic leukemia (ALL), acute myeloid leukemia (AML), non-Hodgkin's lymphoma (NHL), diffuse large cell lymphoma (DLCL), diffuse large B cell lymphoma (DLBCL), Hodgkin's lymphoma, multiple myeloma, renal cell carcinoma (RCC), neuroblastoma, colorectal cancer, bladder cancer, breast cancer, colorectal cancer, ovarian cancer, melanoma, sarcoma, prostate cancer, lung cancer, esophageal cancer, hepatocellular carcinoma, pancreatic cancer, astrocytoma, mesothelioma, head and neck cancer, and medulloblastoma, and liver cancer. In some embodiments, the cancer is selected from a melanoma, head and neck cancer, bladder cancer, lung cancer, cervical cancer, pancreatic cancer, breast cancer, and colorectal cancer. In some embodiments, the cancer is insensitive, or resistant, to treatment with a PD1 inhibitor. In some embodiments, the cancer is insensitive, or resistant to treatment with a PD1 inhibitor and is selected from a melanoma, head and neck cancer, bladder cancer, lung cancer, cervical cancer, pancreatic cancer, breast cancer, and colorectal cancer.

As described above, several immune checkpoint inhibitors are currently approved for use in a variety of oncologic indications (e.g., CTLA4 inhibitors, PD1 inhibitors, PDL1 inhibitors, etc.). In some embodiments, administration of an agent or the combination of an agent and an ICI described herein results in an enhanced therapeutic effect (e.g., a more significant reduction in tumor growth, an increase in tumor infiltration by lymphocytes, an increase in the length of progression free survival, etc.) than is observed after treatment with an immune checkpoint inhibitor alone.

Further, some oncologic indications are non-responsive (i.e., are insensitive) to treatment with immune checkpoint inhibitors. Further still, some oncologic indications that are initially responsive (i.e., sensitive) to treatment with ICIs develop an inhibitor-resistant phenotype during the course of treatment. Therefore, in some embodiments, the agent, or combination of agent and ICI, described herein, or compositions thereof, are administered to treat a cancer that is resistant (or partially resistant) or insensitive (or partially insensitive) to treatment with one or more immune checkpoint inhibitors. In some embodiments, administration of the agent, or combination of agent and ICI, or compositions thereof to a subject suffering from a cancer that is resistant (or partially resistant) or insensitive (or partially insensitive) to treatment with one or more immune checkpoint inhibitors results in treatment of the cancer (e.g., reduction in tumor growth, an increase in the length of progression free survival, etc.). In some embodiments, the cancer is resistant (or partially resistant) or insensitive (or partially insensitive) to treatment with a PD1 inhibitor.

In some embodiments, the agent or compositions thereof is administered in combination with an immune checkpoint inhibitor. In some embodiments, administration of the agent in combination with the immune checkpoint inhibitor results in an enhanced therapeutic effect in a cancer that is resistant, refractory, or insensitive to treatment by an immune checkpoint inhibitor than is observed by treatment with either the agent or the immune checkpoint inhibitor alone. In some embodiments, administration of the agent in combination with the immune checkpoint inhibitor results in an enhanced therapeutic effect in a cancer that is partially resistant, partially refractory, or partially insensitive to treatment by an immune checkpoint inhibitor than is observed by treatment with either the agent or the immune checkpoint inhibitor alone. In some embodiments, the cancer is resistant (or partially resistant), refractory (or partially refractory), or insensitive (or partially insensitive) to treatment with a PD1 inhibitor.

In some embodiments, administration of an agent described herein or composition thereof in combination with an anti-PD1 antibody results in an enhanced therapeutic effect in a cancer that is resistant or insensitive to treatment by the anti-PD1 antibody alone. In some embodiments, administration of an agent described herein or composition thereof in combination with an anti-PD1 antibody results in an enhanced therapeutic effect in a cancer that is partially resistant or partially insensitive to treatment by the anti-PD1 antibody alone.

Cancers that demonstrate resistance or sensitivity to immune checkpoint inhibition are known in the art and can be tested in a variety of in vivo and in vitro models. Further, some colorectal cancers are known to be resistant to treatment with an immune checkpoint inhibitor. Further still, some lymphomas are known to be insensitive to treatment with an immune checkpoint inhibitor such as an anti-PD1 antibody and can be modeled in various models by adoptive transfer or subcutaneous administration of lymphoma cell lines.

### Diagnostic Methods and Applications

The present disclosure further provides, in some embodiments, methods of differentiating between ‘responders’ and ‘non-responders’ to immune checkpoint inhibitor (ICI) therapy. In some embodiments, a subject in need of such differentiation is a cancer patient. The data provided herein demonstrates that certain biomarkers, for example, RGS1, NKG7 and/or CCL5, may be useful for identifying subjects who are likely to respond to ICI therapy, and thus are candidates for ICI therapy.

Some aspects of the present disclosure provide method, comprising selecting a subject (e.g., a human subject) who is a candidate for immune checkpoint inhibitory (ICI) therapy. Non-limiting examples of such therapies are provided elsewhere herein and include anti-PD-L1 and anti-PD-1 monoclonal antibody therapies.

In some embodiments, the method further comprises obtaining a biological sample from the subject. The biological sample may be a blood sample or a tissue sample (e.g., tumor biopsy), for example. Other biological samples are contemplated herein.

In some embodiments, the method further assaying the biological sample for a gene or protein level one or more biomarkers selected from regulator of G protein signaling 1 (RGS1) (NCBI Gene ID: 5996; HGNC: HGNC: 9991), natural killer cell granule protein 7 (NKG7) (NCBI Gene ID: 4818; HGNC: HGNC: 7830), and C—C motif chemokine ligand 5 (CCL5) (Gene ID: 6352; HGNC: HGNC: 10632). In some embodiments, the biomarker assayed is RGS1. In some embodiments, the biomarker assayed is NKG7. In some embodiments, the biomarker assayed is CCL5. In some embodiments, one or more than one biomarker(s) are assayed. For example, two, three, or all four biomarkers may be assayed.

In some embodiments, the methods further comprise comparing the gene or protein level to a control level, wherein the control level is based on a biological sample that is non-responsive to ICI therapy.

In some embodiments, the subject is a cancer patient. For example, the cancer patient may have a basal cell carcinoma, bladder cancer, breast cancer, cervical cancer, colorectal cancer, endometrial cancer, esophageal carcinoma, gastric cancer, head and neck cancer, hepatocellular carcinoma, hodgkin's lymphoma, malignant pleural mesothelioma, melanoma, melanoma, metastatic, merkel cell carcinoma, non-small cell lung cancer, primary mediastinal large b-cell lymphoma, renal cell carcinoma, small cell lung cancer, solid tumors, squamous cell carcinoma, stomach cancer, or a urothelial carcinoma

In some embodiments, the method further comprises diagnosing the subject as a responder to ICI therapy if the gene or protein level of one or more of RGS1, NKG7 and CCL5 is at least 1-fold, 1.1-fold, 1.2-fold, 1.3-fold, 1.4-fold, or 1.5-fold higher than the control level.

In some embodiments, the method further comprises administering an ICI therapy to the subject.

### Compositions and Kits

The present disclosure provides compositions comprising an agent that modifies expression of a gene or function of a product of the gene, wherein the gene is a homolog (e.g., human ortholog) of a gene that maps to a mouse Chr15 QTL, for example.

In some embodiments, the present disclosure provides compositions. In some embodiments, a composition includes an agent (e.g., antibody, small molecule drug, programmable nuclease, or RNAi molecule) that modifies expression of a gene homolog (e.g., human ortholog), or function of a product of the gene homolog (e.g., human ortholog), of a gene that maps to the mouse Chr15 QTL described herein (e.g., GRCm38/mm10 Chr15: 75.79-81.31 Mb, such as GRCm38/mm10 Chr15: 78,175,584-78,274,852 Mb or GRCm38/mm10 Chr15: 79,998,008-80,088,247 Mb).

In some embodiments, a composition includes an inhibitor of gene expression. In some embodiments, a composition includes an inhibitor of protein function. In some embodiments, a composition includes an inhibitor of human NCF4 gene expression. In some embodiments, a composition includes an inhibitor of human NCF4 protein function.

In some embodiments, a composition includes an antibody. For example, a composition may include an anti-NCF4 antibody or an anti-p40-phox antibody.

In some embodiments, a composition includes a small molecule drug inhibitor. For example, a composition may include a small molecule drug inhibitor of human NCF4.

In some embodiments, a composition includes a programmable nuclease. For example, a composition may include a Cas endonuclease and a (one or more) guide RNA that targets (e.g., binds to) a gene, such as a human NCF4 gene or gene variant.

In some embodiments, a composition includes an RNA interference molecule. For example, a composition may include an RNAi molecules that targets a gene, such as a human NCF4 gene or gene variant.

In some embodiments, the composition further comprises an immune checkpoint inhibitor, such as a PD-1, PD-L1 or CTLA-4 inhibitor. In some embodiments, the immune checkpoint inhibitor is an antibody, such an anti-PD-1 antibody, anti-PD-L1 antibody, or anti-CTLA-4 antibody. For example, FDA-approved PD-L1 inhibitors include Atezolizumab (Tecentriq®, Genentech), Avelumab (Bavencio®, Pfizer), and Durvalumab (Imfinzi®, AstraZeneca); FDA-approved PD-1 inhibitors include Pembrolizumab (Keytruda®, Merck) and Nivolumab (Opdivo®, Bristol-Myers Squibb); and FDA-approved CTLA4 inhibitors include Ipilimumab (Yervoy®, Bristol-Myers Squibb). Additional inhibitory immune checkpoint molecules that may be used include A2AR, B7-H3, B7-H4, BTLA, IDO, LAG3 (e.g., BMS-986016, under development by BSM), KIR (e.g., Lirilumab, under development by BSM), TIM3, TIGIT, and VISTA.

A composition refers to a formulation of an agent that is capable of being administered or delivered to a subject. Typically, formulations include all physiologically acceptable compositions including derivatives and/or prodrugs, solvates, stereoisomers, racemates, or tautomers thereof with any physiologically acceptable carriers, diluents, and/or excipients. A “therapeutic composition” or “pharmaceutical composition” (used interchangeably herein) is a composition of agent (e.g., an inhibitor of NCF4 gene expression and/or NCF4 protein function) capable of being administered to a subject for the treatment of a particular cancer.

A pharmaceutically acceptable is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A pharmaceutically acceptable excipient includes without limitation any excipient, adjuvant, carrier, glidant, sweetening agent, diluent, preservative, dye/colorant, flavor enhancer, surfactant, wetting agent, dispersing agent, suspending agent, stabilizer, isotonic agent, solvent, surfactant, and/or emulsifier which has been approved by the United States Food and Drug Administration as being acceptable for use in humans and/or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations. Except insofar as any conventional media and/or agent is incompatible with the agents of the present disclosure, its use in therapeutic compositions is contemplated. Supplementary active ingredients also can be incorporated into the compositions.

A pharmaceutically acceptable salt includes both acid and base addition salts. Pharmaceutically-acceptable salts include the acid addition salts (formed with the free amino groups of the protein) and which are formed with inorganic acids such as, for example, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, ptoluenesulfonic acid, trifluoroacetic acid, undecylenic acid, and the like. Salts formed with the free carboxyl groups can also be derived from inorganic bases such as, for example, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

In some embodiments, the present disclosure provides kits for carrying out a method described herein. In some embodiments, a kit includes an agent (e.g., antibody, small molecule drug, programmable nuclease, or RNAi molecule) that modifies expression of a gene homolog (e.g., human ortholog), or function of a product of the gene homolog (e.g., human ortholog), of a gene that maps to the mouse Chr15 QTL described herein (e.g., GRCm38/mm10 Chr15: 75.79-81.31 Mb, such as GRCm38/mm10 Chr15: 78,175,584-78,274,852 Mb or GRCm38/mm10 Chr15: 79,998,008-80,088,247 Mb).

In some embodiments, a kit includes an inhibitor of gene expression. In some embodiments, a kit includes an inhibitor of protein function. In some embodiments, a kit includes an inhibitor of human NCF4 gene expression. In some embodiments, a kit includes an inhibitor of human NCF4 protein function.

In some embodiments, a kit includes an antibody. For example, a kit may include an anti-NCF4 antibody or an anti-p40-phox antibody.

In some embodiments, a kit includes a small molecule drug inhibitor. For example, a kit may include a small molecule drug inhibitor of human NCF4.

In some embodiments, a kit includes a programmable nuclease. For example, a kit may include a Cas endonuclease and a (one or more) guide RNA that targets (e.g., binds to) a gene, such as a human NCF4 gene or gene variant.

In some embodiments, a kit includes an RNA interference molecule. For example, a kit may include an RNAi molecules that targets a gene, such as a human NCF4 gene or gene variant.

In some embodiments, the kit further comprises an immune checkpoint inhibitor, such as a PD-1, PD-L1 or CTLA-4 inhibitor. In some embodiments, the immune checkpoint inhibitor is an antibody, such an anti-PD-1 antibody, anti-PD-L1 antibody, or anti-CTLA-4 antibody (see above).

Components of a kit can be in separate containers or can be combined in a single container.

In addition to above-mentioned components, in some embodiments a kit further comprises instructions for using the components of the kit to practice the methods of the present disclosure. The instructions for practicing the methods are generally recorded on a suitable recording medium. For example, the instructions may be printed on a substrate, such as paper or plastic, etc. As such, the instructions may be present in the kits as a package insert or in the labeling of the container of the kit or components thereof (i.e., associated with the packaging or sub-packaging). In other embodiments, the instructions are present as an electronic storage data file present on a suitable computer readable storage medium, e.g., CD-ROM, diskette, flash drive, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

The following Examples have been included to illustrate modes of the presently claimed subject matter. Certain aspects of the following Examples are described in terms of techniques and procedures found or contemplated by the present co-inventors to work well in the practice of the presently claimed subject matter. These Examples illustrate standard laboratory practices of the co-inventors. In light of the present disclosure and the general level of skill in the art, those of skill will appreciate that the following Examples are intended to be exemplary only and that numerous changes, modifications, and alterations can be employed without departing from the scope of the presently claimed subject matter.

### Example 1: Profiling CCF1 Lines for Response to Anti-PD-1 Treatment and Determining the Effect of Host Genetics on Immune Checkpoint Inhibitor (ICI) Response

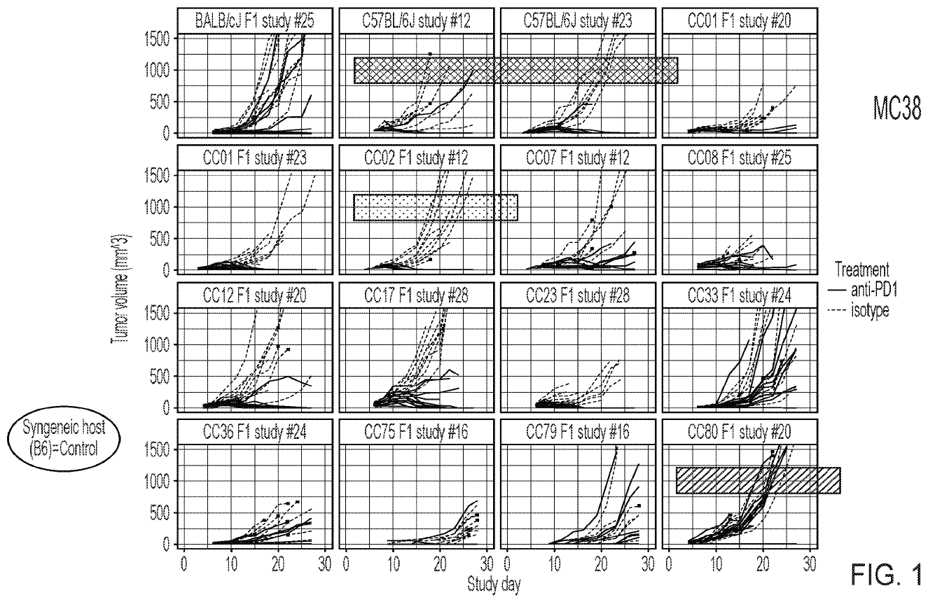

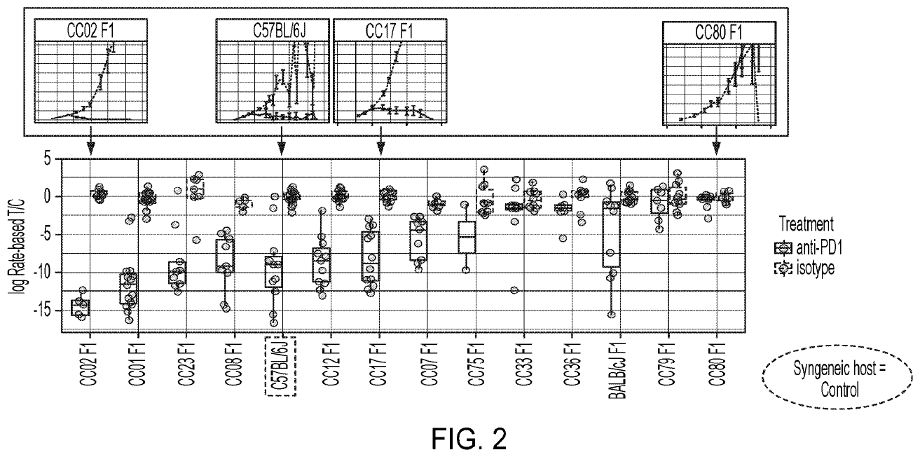

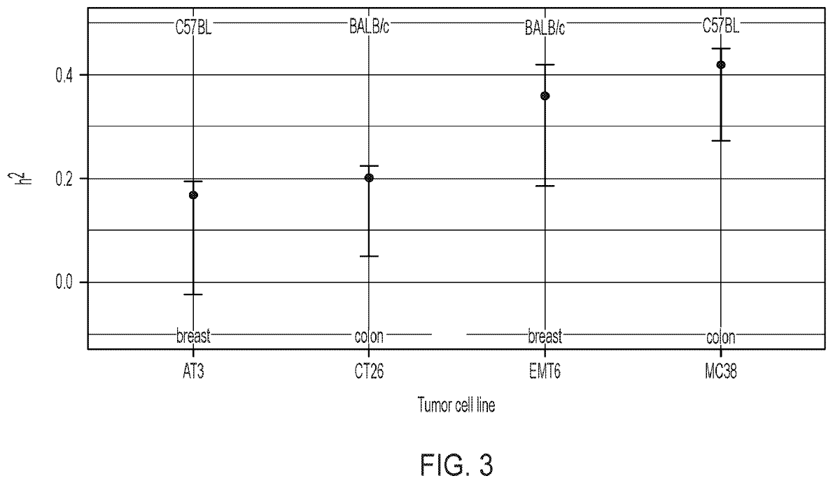

Quantitative and reproducible variation in anti-PD-1 response was observed across CCF1 lines. 33 CCF1 lines were screened in the MC38 tumor model, 19 CCF1 lines in the CT26 model, 16 CCF1 lines in the AT3 model, and 15 CCF1 lines in the EMT6 model in nearly 2,500 mice as seen in Table 1. As shown in FIGS. 1 and 2, there was sufficient strain-level diversity (>10 CCF1 lines) to assess heritability of anti-PD-1 response in four syngeneic tumor lines representing breast and colon cancer types. When the heritability (h²) was calculated using the modified per-mouse rate-based treatment/control metric, three out of the four (CT26, EMT6, and MC38) were statistically significant and ranged from 0.2 to 0.45, as shown in FIG. 3. This data shows that the genetic background of the host significantly affected the anti-PD-1 response.

TABLE 1
Cell Lines Tested in Various Tumor Models

| Cell Line | Cancer Type | Syngeneic Host | # Strains | #Mice |
|---|---|---|---|---|
| AT3 | breast | C57BL | 16 | 360 |
| CT16 | colon | BALB/c | 19 | 538 |
| EMT6 | breast | BALB/c | 15 | 358 |
| MC38 | colon | C57BL | 33 + CCF1N1 | 1,217 |

### Example 2: Genetic Mapping to Identify Loci Contributing to Variation in Anti-PD-1 Response

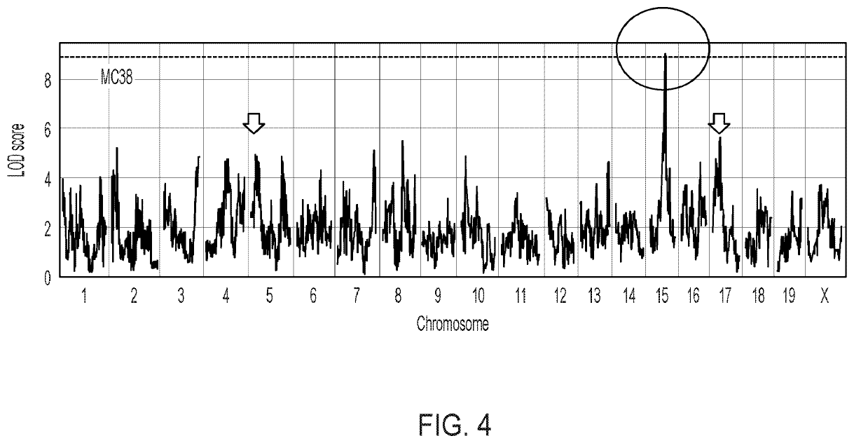

In order to identify genomic loci (quantitative trait loci; QTL) harboring variation that influences anti-PD-1 response, genetic mapping in CCF1 lines was performed. QTL were mapped for responses in all four syngeneic tumor lines (AT3, CT16, EMT6, and MC38) and a significant QTL (permutation P<0.05) for response in MC38 on Chromosome 15 was found, as shown in FIG. 4.

### Example 3: Chr15 QTL Identified in Mapping Anti-PD-1 Response in the MC38 Model

### Ncf4 Gene

The extensive investigation of the Chr15 QTL identified in mapping anti-PD-1 response in the MC38 model was performed. The QTL interval implicated by genetic mapping spanned 5.2 Mb and contained approximately 200 genes.

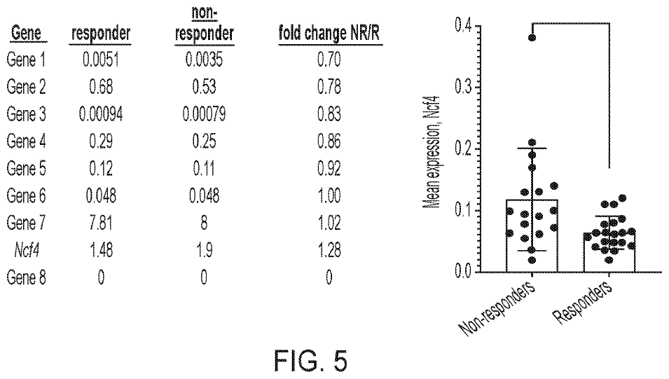

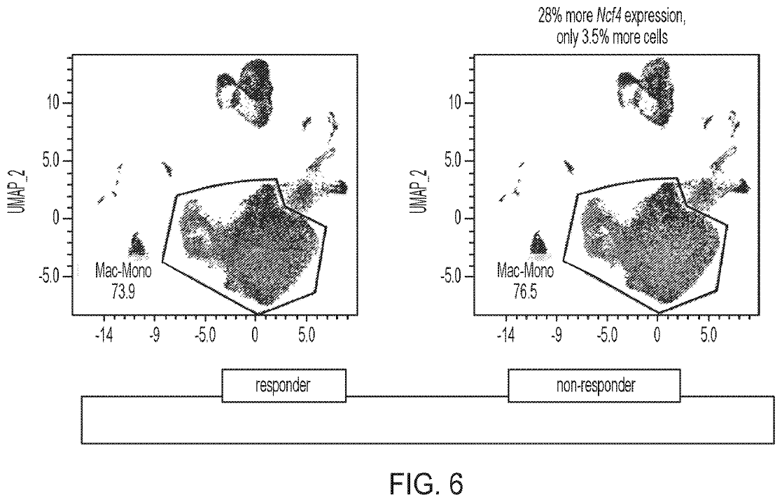

Single cell transcriptomics data (scRNA-Seq) was used to probe the expression of genes located within this Chr15 QTL interval. Only a subset of genes was expressed in the immunocytes, and of these genes Ncf4 was identified as a gene that was among the most highly expressed and having a large difference in expression between non-responder and responder strain tumors (1.3-fold; FIG. 5). This difference in Ncf4 expression appears to be largely driven by a difference in expression within monocytes and macrophages and not due to a greater number of these cells (FIG. 6).

NCF4 is a member of the NADPH oxidase complex, which is a major source of cellular reactive oxygen species. NCF4 has a role in innate immunity and human disease, NADPH oxidase impacts antigen presentation, and NCF4 has associations with kidney and colorectal cancer.

### Ncf4 Gene Variants

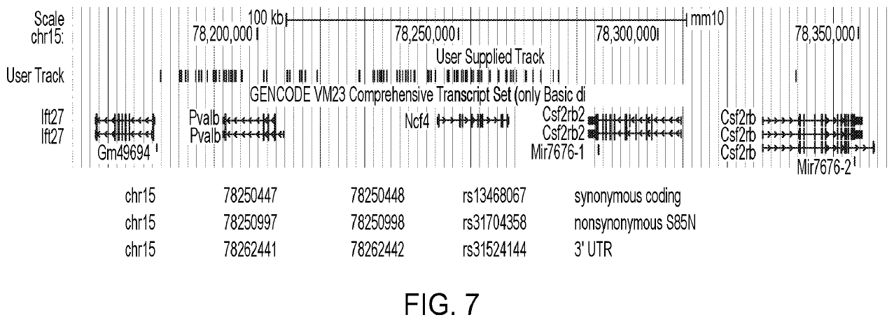

Examination of the estimated effect of a CCF1 strain carrying ancestry from each of the eight CC founder strains at this Chr15 QTL revealed a bifurcation between two groups of strains. CCF1 strains carrying ancestry at the Chr15 QTL from B6, CAST, PWK, or WSB were associated with a better anti-PD-1 response, as measured by the rate-based treatment/control metric (RTC). CCF1 strains carrying ancestry at the Chr15 QTL from any of the remaining CC founder strains (namely 129, A/J, NOD, or NZO) were associated with a poorer anti-PD-1 response. This 4:4 bifurcation suggests that a primary driver of this QTL might be a biallelic variant or set of variants where B6, CAST, PWK, and WSB share one allele and the remaining four founder strains carry the other allele. Of the 74,053 single nucleotide polymorphisms and insertions/deletions segregating in the Collaborative Cross within this Chr15 QTL, only 233 variants match this pattern (“candidate variants”). Many of these 233 variants cluster in an ˜50 kb region near the middle of this QTL (data not shown). Interestingly, this cluster of variants overlaps Ncf4. Three Ncf4 coding variants within this set of candidates were identified, and several additional variants upstream of Ncf4 that lie within open chromatin regions containing computationally predicted motif binding sites as seen in FIG. 7. These variants constitute candidates that may affect gene regulation or protein function of NCF4 and could thereby modulate anti-PD-1 response. A top candidate variant encodes a nonsynonymous substitution in NCF4, S85N and a similar mutation (T85N) has been reported in a human patient with chronic granulomatous disease (CGD) in ClinVar database (NM_000631.5; rs112306225); opposing alleles are present in the responder C57BL/6J and non-responder BALB/cJ lines.

### Example 4: Variation in or Around Ncf4 is Acting Through this Gene to Modulate Anti-PD-1 Response

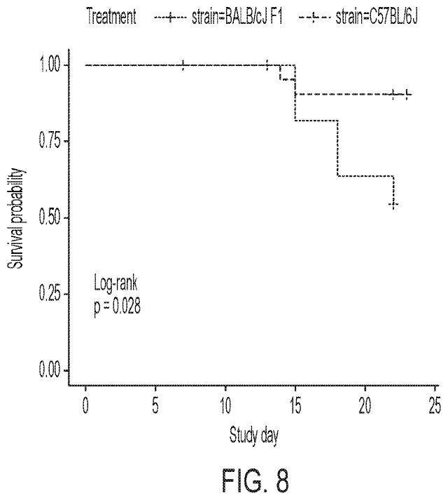

Described herein is compelling indirect evidence from the analysis of the Chr15 QTL that variation in and/or around Ncf4 is acting through this gene to modulate an anti-PD-1 response. Specifically, the responder genotype is associated with the less active Ncf4 allele. This was further confirmed by using BALB/cJ, which harbors the non-responder genotype but was not a founder in the collaborative cross. When F1 crosses between BALB/cJ and C57BL/6J (B6) were produced, the resulting F1 hybrids were found to impair response to anti-PD-1 compared to the B6 responder phenotype (FIG. 8). Table 2 provides data showing that while 82% of B6 mice responded, only 25% of BALB/cJ responded. This means that ancestral origin of the Ncf4 non-responder genotype in BALB/c (and not other segregating genetic variations) was the likely cause. Importantly, functional data suggest that tumor associated immunocytes from mouse strains carrying the responder haplotype at Ncf4 produce less reactive oxygen species, which tips the scale in favor of a better anti-PD-1 response.

TABLE 2
Immunotherapy Response Classification

| | | | | Complete | Partial/ | |
|---|---|---|---|---|---|---|
| Cell line | Strain | Treatment | N | responder | non-responder | % responding |
| MC38 | BALB/cJ F1 | isotype | 12 | 0 | 12 | 0% |
| MC38 | BALB/cJ F1 | anti-PD1 | 12 | 3 | 9 | 25% |
| MC38 | C57BL/6J | isotype | 22 | 0 | 22 | 0% |
| MC38 | C57BL/6J | anti-PD1 | 22 | 18 | 4 | 82% |

In order to provide direct evidence that Ncf4 is the causal gene underlying this QTL, a Ncf4 knockout mouse was engineered using CRISPR/Cas9-mediated disruption by nonhomologous end joining. The strains carrying the responder haplotype at Ncf4 produce less reactive oxygen species in favor of a better anti-PD-1 response. Since the MC38 syngeneic host, C57BL/6J, carries a responder haplotype and inbred C57BL/6J responds very well to anti-PD-1, an Ncf4 knockout on non-responder genetic backgrounds was created. The Ncf4 knockout on the BALB/cJ (non-responder) background was mated to the syngeneic host C57BL/6J (responder). The anti-PD-1 response is being tested in the MC38 model.

### Example 5: Characterization of Immunophenotypes with Anti-PD-1 Response

All samples from MC38 responder (CC75, CC2, CC1) and non-responder strains (CC36, CC79, CC80) necessary for immunophenotyping studies were acquired. In these studies, tumors were grown until palpable (75-110 mm\hat{ }3) before a single dose of αPD-1 or isotype control was administered. 48 hours following this dose, tumors were harvested and banked. This model system has the unique advantage of allowing analysis of the tumor steady state (isotype treated tumors) at an early, immunotherapy responsive time, and following a single dose of anti-PD1.

To minimize batch effects and make the best comparisons possible between responders and non-responders, sets of both responder and non-responder samples for assays were processed at the same time.

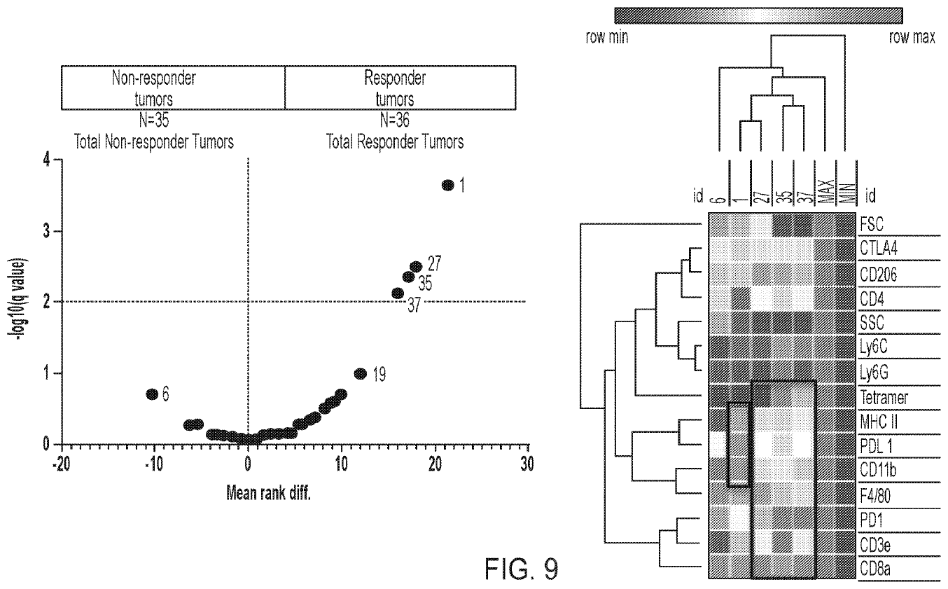

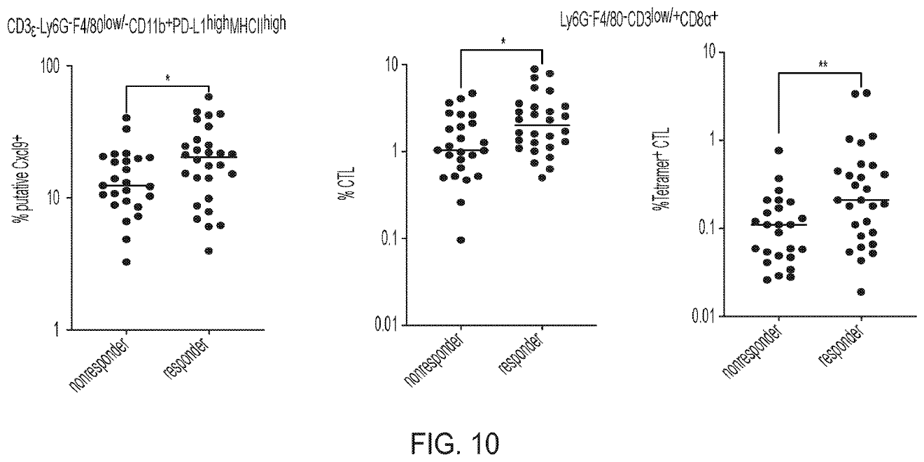

Three main assays for immunophenotyping were used including: FACS, single cell (sc) RNAseq, and bulk RNAseq. As shown in FIGS. 9 and 10, responder strain tumors had significantly higher levels of total and MC38-specific, tetramer-stained cytotoxic lymphocytes (CTL) than non-responder tumors. FLOWSOM analysis and a separate manual gating analysis also demonstrated enrichment of a PD-L1^{high}MHCII^{high} macrophage population in responder tumors. These results suggest that responder tumors contain PD-L1⁺ macrophages inhibiting CTL through PD-1-PD-L1 interactions.

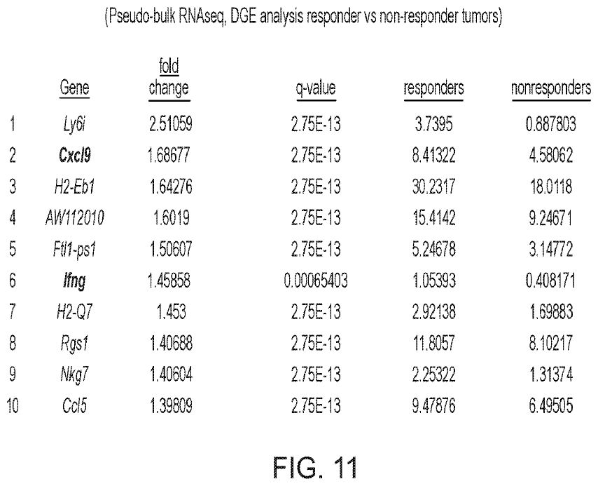

The sc-RNAseq experiment yielded a dataset of approximately 95,000 cells from a total of 47 responder and non-responder MC38 tumors. Tumor immune cell infiltrates were composed primarily of monocyte/macrophages followed by T and NK cells. As shown in FIG. 11, a pseudobulk RNAseq analysis comparing responder to non-responder tumors revealed gene expression that confirms the translational relevance of our model (Cxc19, Ifng, H2-Eb1 and H2-Q7) as well as provides novel biomarkers potentially useful for predicting whether a patient will respond to immunotherapy before treatment. These markers include AW112010, Rgs1, Nkg7, and Ccl5. Ftl1-ps1 and Ly6i were found to be a pseudogene and mouse specific marker, respectively.

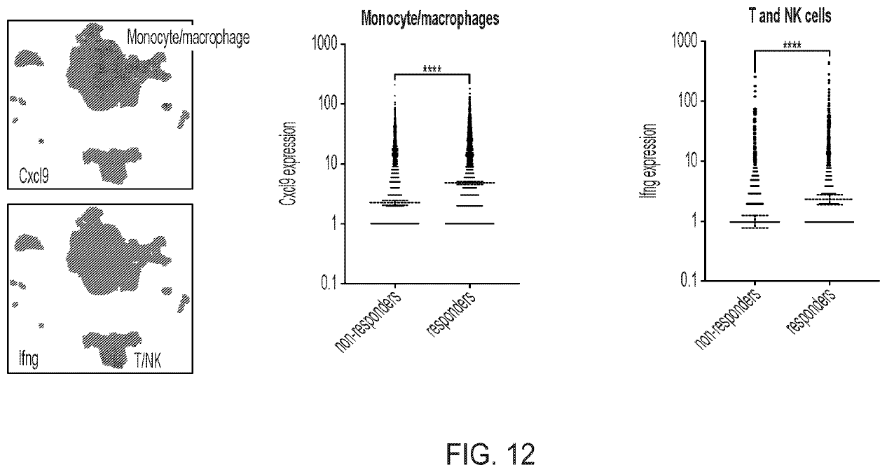

The expression of the IFNγ inducible chemokine CXCL9 has recently been shown to be one of the strongest predictors of ICI response in patients. IFNγ has long been demonstrated to be necessary for anti-tumor immune responses. Cxc19 expression is largely confined to a subset of cells in the monocyte/macrophage cluster, whereas Ifng expression was confined to a subset of cells in the T/NK cell cluster. Comparing responder tumors to non-responder tumors, responder tumors had a significantly higher magnitude of Cxcl9 and Ifng expression as seen in FIG. 12.

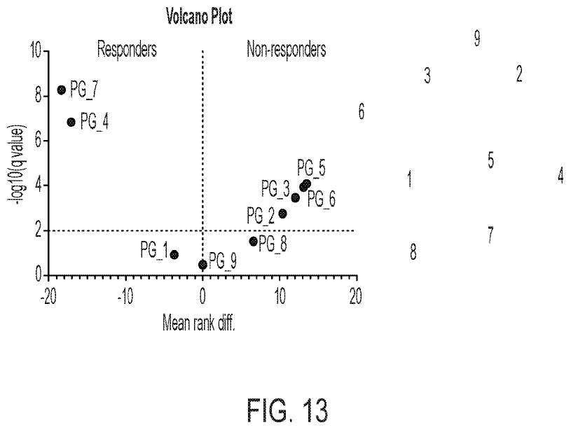

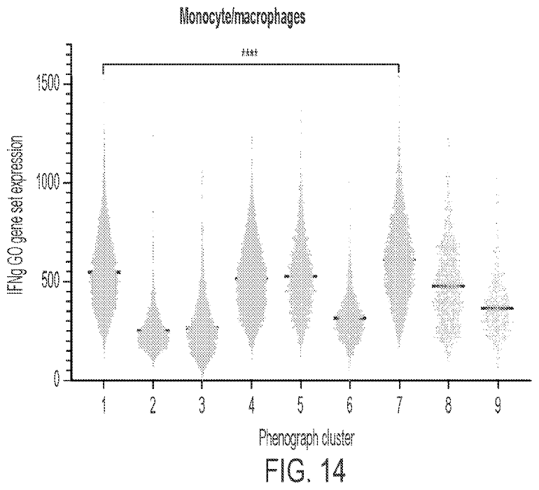

Comparing the intratumor frequencies of monocyte/macrophage clusters between responder and non-responder tumors, 2 subclusters (subclusters 4 and 7), expressing the highest levels of Cxc19, were found to be significantly enriched in responder tumors as shown in FIG. 13. Because Cxc19 is induced by IFNγ, we determined which subclusters were responding to IFNγ by examining the expression of genes taken from a response to IFNγ GO gene set. Subcluster 7 was determined to have the highest IFNγ GO gene set expression indicating this subcluster was being stimulated by IFNγ as seen in FIG. 14. To understand the relationship between responder enriched subclusters 4 and 7, a monocle pseudotime analysis was performed. This analysis demonstrated that subclusters 4 and 7 represent two differentiation extremes (data not shown). Collectively, these data suggest that in responder tumors IFNγ secreted by CTL stimulates the PD-L1^{high} subcluster 7 to differentiate into Cxc19^{high} subcluster 4, setting the stage for response to ICI.

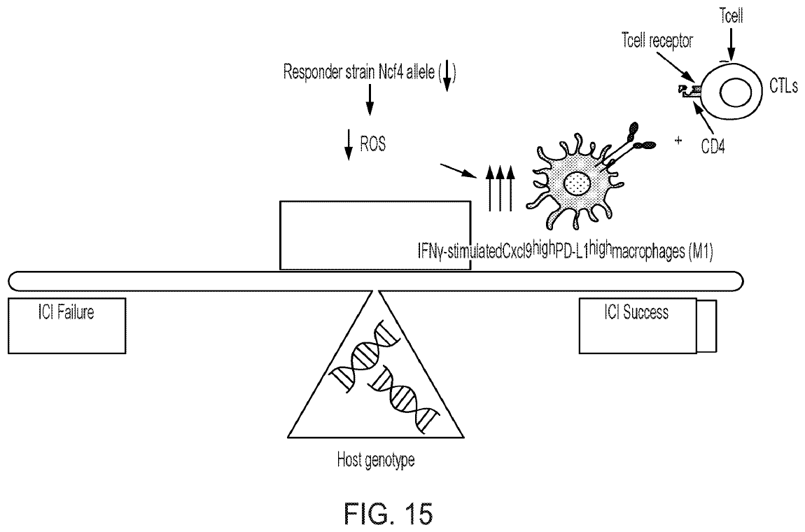

The frequencies of CTL and Cxcl9-expressing macrophages define tumors that are poised to respond to ICI. Responder tumors have significantly higher expression of Ifng and Cxcl9, markers that have been demonstrated to be biomarkers of ICI response in the clinic. The data provided herein suggests that the NADPH oxidase gene Ncf4 impacts the intratumor redox environment: Ncf4 alleles that decrease ROS production by NADPH oxidase promote the differentiation of Cxc19+ macrophages over M2-type tumor associated macrophages. These Cxc19+ macrophages recruit CTL into tumors through the actions of CXCL9 and create an inflammatory environment beneficial to anti-tumor CTL responses (FIG. 15).

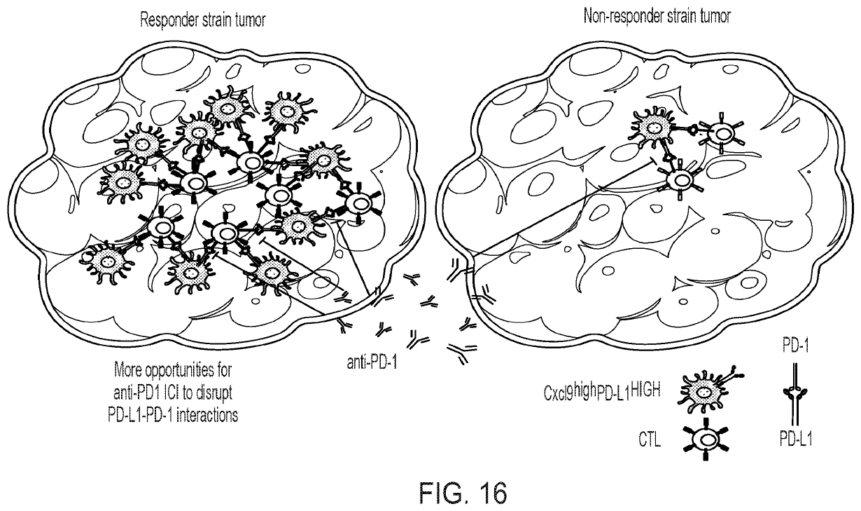

In order to further investigate how cell-cell interactions influence ICI response versus non-response, spatial transcriptomics on the 10× Genomics Visium platform was used to deconstruct the complexity of cellular crosstalk between the tentatively identified Cxc19^{high} monocyte/macrophage population, dendritic cells and CTLs. It was hypothesized that MC38 tumors from responder CC-F1 strains will contain greater numbers of co-localized Cxc19^{high} monocyte/macrophages and CTL than non-responder strains (FIG. 16).

To address this hypothesis, the MC38 IO model was used to profile tumors of two isotype-treated tumor samples from responder (CC075 F1) and non-responder (CC080 F1) strains (four tumors total). To study tumors of a size capable of responding to ICI, female mice received a single dose of isotype control when tumors were palpable (75-110 mm3), before harvest two days later. Characterization of early cellular and spatial heterogeneity of tumors by limiting the tumor size dosing window. It is likely that intratumor immune configurations established early in tumor growth will impede the effect of ICI on non-responder tumors while poising responder tumors for regression.

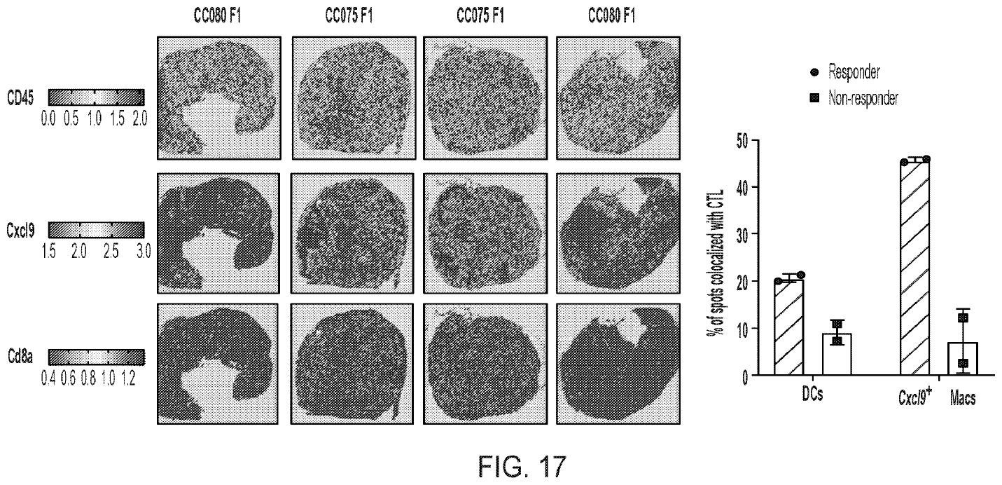

The expression of Ptprc (CD45), a pan-immune marker, suggested that both responder and non-responder tumors were similarly infiltrated (disperse infiltration, no clear punctate foci of immune cells) by immune cells (FIG. 17). In contrast, examining the monocyte/macrophage response marker Cxcl9, responder tumors displayed substantially more positive spots with clear punctate foci, suggesting intratumor clusters of Cxcl9^{high} monocyte/macrophages. An examination of the CTL marker, Cd8a, revealed a similar pattern to Cxcl9. By analyzing Ptprc⁺ spots co-expressing Adgrel, Itgam, and Cxcl9 (macrophages) or Batf3 and/or Zbtb46 (dendritic cells) with spots also expressing the CTL markers Thy1 and Cd8a, it was found that Cxcl9⁺ macrophages were the predominant antigen-presenting cell population co-localizing with CTL in responder strain tumors (responder strain tumors have >4× CTL-macrophage co-localized spots than non-responder strain tumors (FIG. 17). Furthermore, though fewer in number than macrophage-CTL colocalized spots, dendritic cells colocalized with CTL around 2× greater in responder strain tumors than in non-responders. These data support the model in FIG. 16 and are consistent with the hypothesis that genetic variation around the Ncf4 gene may poise tumors to respond to PD-1 blockade by increasing the numbers of intratumor Cxcl9⁺ macrophages and CTLs in tumors.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between and including the upper and lower ends of the range are specifically contemplated and described herein.

What is claimed:

1. A method, comprising administering to a cancer patient an agent that modifies expression of a gene or function of a product of the gene, wherein the gene is a human ortholog of a gene that maps to a mouse Chromosome 15 quantitative trait locus (QTL).

2. A method, comprising:
(a) assaying a biological sample from a cancer patient for presence of a gene or a product of the gene, or a variant of the gene or product of the gene, wherein the gene maps to a Chromosome 15 quantitative trait locus (QTL); and
(b) administering to the cancer patient an agent that modifies expression of the gene or function of the product of the gene, or modifies expression of the variant of the gene or function of the variant of the product of the gene.

3. The method of claim 2 further comprising administering to the cancer patient an immune checkpoint inhibitor.

4. A method, comprising:
(a) administering to a cancer patient an agent that modifies expression of a gene or function of the gene product, wherein the gene maps to a Chromosome 15 quantitative trait locus (QTL); and
(b) administering to the cancer patient an immune checkpoint inhibitor.

5. The method of any one of the preceding claims, wherein the biological sample is a blood sample, optionally comprising monocytes and macrophages.

6. The method of any one of the preceding claims, wherein the agent is administered only if expression of the gene or function of the gene is higher.

7. The method of claim 6, wherein the agent is administered only if expression of the gene or function of the gene is higher in monocytes and macrophages, relative to a control.

8. The method of any one of the preceding claims, wherein the agent inhibits expression of the gene or function of the product of the gene, or inhibits expression of the variant of the gene or function of the variant of the product of the gene.

9. The method of claim 8, wherein the agent targets monocytes and macrophages.

10. The method of any one of the preceding claims, wherein the Chromosome 15 QTL comprises an NCF4 gene.

11. The method of claim 10, wherein the Chromosome 15 QTL spans a chromatin region within 2 megabases (Mb) upstream from and 2 Mb downstream from the NCF4 gene.

12. The method of claim 11, wherein the gene is an NCF4 gene or NCF4 gene variant.

13. The method of claim 12, wherein the NCF4 gene variant encodes an NCF4 protein variant that comprises a mutation corresponding to an S85N mutation in a wild-type mouse NCF4 protein.

14. The method of claim 9, wherein the gene is a CSF2RB gene or a CSF2RB gene variant.

15. The method of claim 9, wherein the gene is a PVALB gene or a PVALB gene variant.

16. The method of any one of claims 8-15, wherein the agent is an inhibitor of NCF4, CSF2RB, or PVALB gene expression and/or NCF4, CSF2RB, or PVALB protein function.

17. The method of claim 11, wherein the agent is a direct inhibitor of NCF4, CSF2RB, or PVALB gene expression and/or NCF4, CSF2RB, or PVALB protein function.

18. The method of claim 10, wherein the agent is an anti-p40-phox antibody.

19. The method of any one of the preceding claims, wherein the agent is selected from antibodies, small molecule drug, and gene editing molecules.

20. The method of any one of the preceding claims, wherein the immune checkpoint inhibitor is selected from programmed cell death protein 1/programmed cell death ligand 1 (PD-1/PD-L1) inhibitors, cytotoxic T lymphocyte-associated antigen-4 (CTLA-4) inhibitors, lymphocyte activation gene-3 (LAG-3) inhibitors, T cell immunoglobulin and mucin-domain containing-3 (TIM-3) inhibitors, T cell immunoglobulin and ITIM domain (TIGIT) inhibitors, and V-domain Ig suppressor of T cell activation (VISTA) inhibitors.

21. The method of claim 20, wherein the immune checkpoint inhibitor is a PD-1/PD-L1 inhibitor.

22. The method of claim 21, wherein the PD-1/PD-L1 inhibitor is an anti-PD-1 antibody or an anti-PD-L1 antibody.

23. The method of any one of the preceding claims, wherein the cancer patient has a basal cell carcinoma, bladder cancer, breast cancer, cervical cancer, colorectal cancer, endometrial cancer, esophageal carcinoma, gastric cancer, head and neck cancer, hepatocellular carcinoma, Hodgkin's lymphoma, malignant pleural mesothelioma, melanoma, melanoma, metastatic, merkel cell carcinoma, non-small cell lung cancer, primary mediastinal large b-cell lymphoma, renal cell carcinoma, small cell lung cancer, solid tumors, squamous cell carcinoma, stomach cancer, or a urothelial carcinoma.

24. A method, comprising:
selecting a subject who is a candidate for immune checkpoint inhibitory (ICI) therapy;
obtaining a biological sample from the subject;
assaying the biological sample for a gene or protein level one or more biomarkers selected from RGS1, NKG7 and CCL5; and
comparing the gene or protein level to a control level, wherein the control level is based on a biological sample that is non-responsive to ICI therapy.

25. The method of claim 24, wherein the subject is a cancer patient.

26. The method of claim 24 or 25, wherein the ICI therapy is anti-PD-L1 or anti-PD-1 antibody therapy.

27. The method of any one of claims 24-26, wherein the biological sample is a blood sample.

28. The method of any one of claims 24-27, wherein the method further comprises diagnosing the subject as a responder to ICI therapy if the gene or protein level of one or more of RGS1, NKG7 and CCL5 is at least 1-fold, 1.1-fold, 1.2-fold, 1.3-fold, 1.4-fold, or 1.5-fold higher than the control level.

29. The method of claim 28, further comprising administering an ICI therapy to the subject.
